(19) United States
(12) Patent Application Publication
(10) Pub. No.: US 2018/0221435 A1
(43) Pub. Date: Aug. 9, 2018

(54) ITCH TREATMENT USING A COMBINATION OF NEUROKININ-1, GASTRIN RELEASING PEPTIDE, AND GLUTAMATE RECEPTOR ANTAGONISTS

(71) Applicant: The Regents of the University of California, Oakland, CA (US)

(72) Inventors: Earl Carstens, Davis, CA (US); Tasuku AKIYAMA, Davis, CA (US); Mitsutoshi TOMINAGA, Chiba (JP)

(21) Appl. No.: 15/872,811

(22) Filed: Jan. 16, 2018

Related U.S. Application Data
(63) Continuation of application No. 14/650,797, filed on Jun. 9, 2015, now abandoned.
(60) Provisional application No. 61/753,800, filed on Jan. 17, 2013.

(51) Int. Cl. A61K 38/10; A61K 31/451; A61K 45/06; A61K 31/498

(52) U.S. Cl. CPC A61K 38/105; A61K 31/451; A61K 2300/00; A61K 31/498; A61K 45/06

(57) ABSTRACT

Methods, and compositions are provided for inhibition of histamine and non-histamine dependent itch signal transmission or scratch behavior. In one aspect, the present invention further comprises administering to the subject an inhibitor of histamine-dependent itch signal transmission. In some cases, the inhibitor of histamine independent itch signal transmission comprises an NK-1 receptor antagonist or the inhibitor of histamine independent itch signal transmission comprises a GRP receptor antagonist. In some cases, the method comprises administering two inhibitors of histamine independent itch signal transmission. For example, the inhibitors of histamine independent itch signal transmission can comprise an NK-1 receptor antagonist and a GRP receptor antagonist. In another embodiment, the invention provides a method of treating itch comprising administering to a subject suffering from itch an NK-1 receptor antagonist, a GRP receptor antagonist, and an AMPA receptor antagonist.

### CROSS-REFERENCE TO RELATED APPLICATION

The present application is a Continuation of U.S. application Ser. No. 14/650,797, filed Jun. 9, 2015, which is a U.S. National Phase of PCT/US2014/011839, International Filing Date of Jan. 16, 2014, which claims priority to U.S. Provisional Application No. 61/753,800, filed Jan. 17, 2013, the contents of which are hereby incorporated by reference in the entirety for all purposes.

### STATEMENT AS TO RIGHTS TO INVENTIONS MADE UNDER FEDERALLY SPONSORED RESEARCH AND DEVELOPMENT

The invention described and claimed herein was made utilizing funds supplied by the United States National Institute of Health under contract numbers DE013685 and AR057194 and AR063228. The government has certain rights in this invention.

### BACKGROUND OF THE INVENTION

Chronic itch is a burdensome clinical problem that decreases the quality of life (Weisshaar et al., 2006), yet the neuronal mechanisms of itch are still not fully understood. Recent studies have implicated histamine-dependent and histamine-independent pathways in transmitting itch. The histamine-independent itch pathway involves members of the family of over 50 Mas-related G-protein coupled receptors (Mrgprs), in particular MrgprAs, MrgprB4-5, MrgprC11 and MrgprD, which are restricted to small diameter dorsal root ganglion (DRG) neurons in mice (Dong et al., 2001). Chloroquine and the bovine adrenal medulla peptide 8-22 (BAMS-22) elicit itch-related scratching through MrgprA3 and MrgprC11, respectively, in mice (Liu et al., 2009), and both chloroquine and BAMS-22 elicit itch in humans (Abila et al., 1994; Sikand et al., 2011).

Studies suggest that the roles for substance P (SP) and gastrin releasing peptide (GRP) in the spinal transmission of itch signals and in general are not completely understood. A GRP receptor antagonist partially reduced scratching elicited by a protease-activated receptor type 2 (PAR-2) agonist, compound 48/80 and chloroquine (Sun and Chen, 2007), while GRP receptor knockout mice did not exhibit a reduction in scratching evoked by histamine, serotonin (5-HT) or endothelin-1 (Sun et al., 2009). It was reported that GRP is not essential to activate the GRP receptor, which heterodimerizes with the μ-opioid receptor isoform MOR1D in mouse superficial dorsal horn neurons and may mediate opioid-induced itch (Liu et al., 2011). An antagonist of the neurokinin-1 (NK-1) receptor for SP suppressed scratching elicited by trypsin (Costa et al., 2008), while deletion of the preprotachykinin A gene that encodes SP and neurokinin A in mice did not reduce 5-HT-evoked scratching (Cuellar et al., 2003). Finally, a recent electrophysiological study suggests that glutamate acts as a neurotransmitter for GRP-sensitive spinal neurons (Koga et al., 2011).

### BRIEF SUMMARY OF THE INVENTION

In one embodiment, the present invention provides a method of treating itch comprising administering to a subject suffering from itch an inhibitor of histamine-independent itch signal transmission.

In one aspect, the present invention further comprises administering to the subject an inhibitor of histamine-dependent itch signal transmission.

In some cases, the inhibitor of histamine independent itch signal transmission comprises an NK-1 receptor antagonist or the inhibitor of histamine independent itch signal transmission comprises a GRP receptor antagonist.

In some cases, the method comprises administering two inhibitors of histamine independent itch signal transmission. For example, the inhibitors of histamine independent itch signal transmission can comprise an NK-1 receptor antagonist and a GRP receptor antagonist.

In one aspect, the method the inhibitor of histamine dependent signal transmission is an AMPA receptor antagonist.

In some cases, the step of administering comprises systemic, epidural, or intrathecal administration. Systemic administration can comprise intraperitoneal, subcutaneous, intravenous, oral, intradermal, or dermal administration.

In another embodiment, the invention provides a method of treating itch comprising administering to a subject suffering from itch an NK-1 receptor antagonist, a GRP receptor antagonist, and an AMPA receptor antagonist.

In yet another embodiment, the invention provides a formulation comprising: a histamine independent itch signal transmission inhibitor; a histamine dependent itch signal transmission inhibitor; and a pharmaceutically acceptable excipient.

In some cases, the histamine independent itch signal transmission inhibitor is an NK-1 receptor antagonist. In some cases, the histamine independent itch signal transmission inhibitor is a GRP receptor antagonist.

In one aspect, the histamine dependent itch signal transmission inhibitor comprises an AMPA receptor antagonist.

In another aspect, the present invention provides a formulation that further comprises a second inhibitor of histamine independent itch signal transmission.

In some cases, the inhibitors of histamine independent itch signal transmission are an NK-1 receptor antagonist and a GRP receptor antagonist.

### BRIEF DESCRIPTION OF THE DRAWINGS

FIGS. 1A-1K Combined effects of NK-1, GRP, and AMPA/kainate receptor antagonists on id chloroquine-evoked activity of superficial dorsal horn neurons.

FIG. 1A: Individual example (vehicle control). Peristimulus-time histogram (PSTH; bins: 1s) shows response of superficial dorsal horn neuron to id chloroquine (at arrow). Chloroquine was injected id in the hindpaw receptive field area shown in the upper inset. Lower inset shows superficial recording site (dot). Vehicle (saline) was superfused over spinal cord (bar) during the initial part of the neuronal response to chloroquine. FIG. 1B: As in FIG. 1A for a combination of NK-1, AMPA/kainate and GRP receptor antagonists (PSTH for different superficial dorsal horn unit than in FIG. 1A). Chloroquine was injected id (at arrow) in hindpaw receptive field (upper inset). Shortly thereafter, a combination of NK-1, AMPA/kainate and GRP receptor antagonists was superfused over the spinal cord (bar), resulting in marked suppression of chloroquine-evoked firing. FIG. 1C: Vehicle control. Graph plots mean responses (impulse frequency averaged over 20 sec) of superficial dorsal horn units before (Pre-) and after id chloroquine. Graphs are aligned at time 0 with the onset of spinal superfusion (horizontal bar above graph). Time point −20 represents mean response following id chloroquine, measured 20 seconds prior to onset of spinal superfusion. Error bars: SEM. *: significantly different compared to Pre (p<0.05; Bonferroni-test following one way repeated-measures ANOVA). D: GRP receptor antagonist RC-3095 (20 μM). Format and symbols as in FIG. 1C. #: significantly different compared to chloroquine-evoked response prior to spinal superfusion (p<0.05; Bonferroni-test following one way repeated-measures ANOVA). FIG. 1E: NK-1 receptor antagonist L-733060 (200 μM). Format and symbols as in FIG. 1C. FIG. 1F: Glutamate receptor antagonist CNQX (100 μM). Format and symbols as in FIG. 1C. FIG. 1G: Combined NK-1 and AMPA/kainate receptor antagonists. Format and symbols as in FIG. 1C. FIG. 1H: Combined NK-1, AMPA/kainate and GRP receptor antagonists. Format and symbols as in FIG. 1C. I: Summary of antagonist effects. Bar graph plots mean responses during the 40-60 second period of spinal superfusion of vehicle (black bar), GRP receptor antagonist RC-3095 (gray bar), NK-1 antagonist L-733060 (horizontal striped bar), AMPA/kainate receptor antagonist CNQX (vertical striped bar), L-733060+CNQX (checkered bar) or RC-3095+L-733060+CNQX (white bar). All responses are normalized to the firing rate 20 seconds prior to vehicle or antagonist application. Error bars: SEM. *, significantly different from vehicle group, p<0.05, One-way ANOVA, Bonferroni post-test. #: significantly different from vehicle group, p<0.01, One-way ANOVA, Bonferroni post-test, n=23-35/group. FIG. 1J: Summary of antagonist effects for NS and WDR cells. Bar graph plots mean responses of WDR (black bars) and NS units (white bars) during the 40-60 second period of spinal superfusion of, from left to right, RC-3095 (GRP receptor antagonist), L-733060 (NK-1 antagonist) or CNQX (AMPA/kainate receptor antagonist). All responses are normalized to the firing rate 20 seconds prior to vehicle or antagonist application. Dashed line: mean response during 40-60 second period of spinal superfusion of vehicle. Error bars: SEM. *, significantly different from vehicle group, p<0.05, unpaired t-test., n=9-14/group. FIG. 1K: Recording sites compiled on representative lumbar spinal cord section.

FIGS. 2A-2H Effects of NK-1, GRP, or AMPA/kainate receptor antagonist on id histamine-evoked activity of superficial dorsal horn neurons. FIG. 2A: Individual example (vehicle control). PSTH (bins: 1s) shows response of superficial dorsal horn neuron to histamine (at arrow) injected id in the hindpaw receptive field (upper inset). Lower inset shows superficial recording site (dot). Vehicle (saline) was superfused over spinal cord (bar) during the initial part of the neuronal response to histamine. FIG. 2B: As in FIG. 2A for CNQX (PSTH for a different neuron than in A). Histamine was injected id (at arrow) in hindpaw receptive field (upper inset). Shortly thereafter, CNQX was superfused over the spinal cord (bar), resulting in marked suppression of histamine-evoked firing. FIG. 2C: Vehicle controls. Graph plots mean responses (impulse frequency averaged over 20 sec) of superficial dorsal horn units before (Pre-) and after id histamine (format as in FIG. 4C). FIG. 2D: as in FIG. 2C for spinal superfusion of GRP antagonist RC3095. FIG. 2E: As in FIG. 2C for L-733060. *: significantly different compared to the pre (p<0.05; Bonferroni-test following one way repeated-measures ANOVA). FIG. 2F: As in FIG. 2C for CNQX. #: significantly different compared to histamine-evoked response prior to spinal superfusion (p<0.05; Bonferroni-test following one way repeated-measures ANOVA). FIG. 2G: Summary of antagonist effects. Bar graph plots, from left to right, the mean histamine-evoked responses during the 40-60 second period of spinal superfusion of vehicle (black bar), GRPR antagonist, RC-3095 (gray bar), NK1R antagonist, L-733060 (horizontal striped bar) or AMPA/kainate receptor antagonist, CNQX (white bar). Responses are normalized to the firing rate before the antagonist application. Error bars: SEM. *, significantly different from vehicle group, p<0.05, One-way ANOVA, Bonferroni post-test, n=14-18/group.

FIG. 2H: Summary of antagonist effects for NS and WDR cells. Bar graph plots mean responses of WDR (black bars) and NS units (white bars) during the 40-60 second period of spinal superfusion of, from left to right, RC-3095 (GRP receptor antagonist), L-733060 (NK-1 antagonist) or CNQX (AMPA/kainate receptor antagonist). Dashed line indicates mean responses during the 40-60 second period of spinal superfusion of vehicle. All responses are normalized to the firing rate 20 seconds prior to vehicle or antagonist application. Error bars: SEM. *, significantly different from vehicle group, p<0.05, unpaired t-test., n=6-10/group. Inset: Recording sites.

FIGS. 3A-3G Combined effects of NK-1 and AMPA/kainate receptor antagonists on topical allyl isothiocyanate (AITC)-evoked activity of superficial dorsal horn neurons. FIG. 3A: Individual example shows PSTH of superficial dorsal horn unit's response to topical application of AITC to hindpaw receptive field (upper left inset). Vehicle (saline) was superfused during AITC-evoked response. FIG. 3B: As in FIG. 3A for different superficial dorsal horn neuron, with spinal superfusion of combined NK-1 (L733060) and AMPA/kainate (CNQX) receptor antagonists. FIG. 3C: Vehicle control. Graph plots mean responses (impulse frequency averaged over 20 seconds) of superficial dorsal horn units before (Pre-) and after topical AITC (format as in FIG. 4C). Error bars: SEM. *: significantly different compared to Pre (p<0.05; Bonferroni-test following one way repeated-measures ANOVA). FIG. 3D: as in C for spinal superfusion of NK-1 receptor antagonist. FIG. 3E: as in C for spinal superfusion of AMPA/kainate antagonist. #: significantly different compared to AITC-evoked response prior to spinal superfusion (p<0.05; Bonferroni-test following one way repeated-measures ANOVA). FIG. 3F: as in FIG. 3C for spinal superfusion of combined NK-1 and AMPA/kainate antagonists. #: significantly different compared to AITC-evoked response prior to spinal superfusion (p<0.05; Bonferroni-test following one way repeated-measures ANOVA). FIG. 3G: Summary of antagonist effects. Bar graph plots the mean AITC-evoked responses during the 40-60 second period of spinal superfusion of vehicle (black bar), NK-1 receptor antagonist (gray bar), AMPA/kainate receptor antagonist (horizontal striped bar), or combined NK-1 and AMPA/kainate receptor antagonists (white bar). Responses are normalized to the firing rate before the antagonist application. Error bars: SEM. *, significantly different from vehicle group, p<0.05, One-way ANOVA, Bonferroni post-test #, significantly different from vehicle group, p<0.001, One-way ANOVA, Bonferroni post-test, n=9-26/group.

FIGS. 4A-4F Effects of NK-1 antagonist, CNQX, and both, on noxious pinch-evoked activity of superficial dorsal horn neurons. FIG. 4A: Average responses to pinch. Averaged PSTHs (bins: 1s) show, from left to right, the mean pinch-evoked responses before, during, and after the spinal superfusion of NK-1 receptor antagonist L733060. Error bars: SEM. n=22/group. FIG. 4B: Summary of antagonist effects. Bar graph plots, from left to right, the mean pinch-evoked peak responses before (black bar), during (white bar), and after (black bar) the spinal superfusion of NK-1 receptor antagonist. n=22/group. FIG. 4C: As in FIG. 4A for CNQX. Error bars: SEM. n=39/group. FIG. 4D: As in FIG. 4B for CNQX. Error bars: SEM. *, significantly different from pre group, p<0.001, One-way repeated measures ANOVA, Bonferroni post-test, n=39/group. FIG. 4E: As in FIG. 4A for combined NK-1 antagonist and CNQX. Error bars: SEM. n=54/group. FIG. 4F: As in FIG. 4B for combined NK-1 antagonist and CNQX. Error bars: SEM. *, significantly different from pre group, p<0.001, One-way repeated measures ANOVA, Bonferroni post-test, n=45/group. Inset above shows recording sites for units identified using pinch search stimulus.

FIG. 5. Combined effects of of intrathecally-administered NK-1, GRP, and AMPA/kainate receptor antagonists on chloroquine-elicited scratching. A. Bar graph plots, from left to right, the mean number of scratch bouts/30 minutes elicited by id chloroquine 5 minutes after prior intrathecal injection of vehicle (saline; black bar), the GRP receptor antagonist, RC-3095 (0.3 nmol; gray bar), the NK-1 antagonist L-733060 (22.7 nmol; horizontal striped bar), the AMPA/kainate receptor antagonist, CNQX (20 nmol; vertical striped bar), L-733060+CNQX (checkered bar) or RC-3095+L-733060+CNQX (white bar). Error bars: SEM. *, significantly different from vehicle group, p<0.001, One-way ANOVA, Bonferroni post-test, n=6/group. #, significantly different from vehicle group, p<0.005, One-way ANOVA, Bonferroni post-test, n=6/group. $, significantly different from CNQX group, p<0.05, One-way ANOVA, Bonferroni post-test, n=6/group. Data for NK-1 (striped bar) and GRPR (gray bar) antagonists adapted from [Akiyama T et al., Journal of neurophysiology 109(3):742-748 (2013)].

FIG. 6. Effect of intrathecally-administered AMPA/kainate receptor antagonist on histamine-elicited scratching. A. Bar graph plots, from left to right, the mean number of scratch bouts/30 minutes elicited by id histamine 5 minutes after prior intrathecal injection of vehicle (saline; black bar), the GRP receptor antagonist RC-3095 (0.3 nmol; gray bar), the NK-1 receptor antagonist L-733060 (22.7 nmol; horizontal striped bar) or the AMPA/kainate receptor antagonist CNQX (20 nmol; white bar). Error bars: SEM. *, significantly different from vehicle group, p<0.05, One-way ANOVA, Bonferroni post-test, n=6/group. Data for effects of NK-1 (striped bar) and GRPR antagonist (gray bar) adapted from [Akiyama T et al., Journal of neurophysiology 109(3):742-748 (2013)].

FIGS. 7A-7J Pruritogen-responsive DRG cells double- and triple-labeled for SP-, GRP- and VGLUT2-immunoreactivity. FIG. 7A: Graph plots 340/380 nm ratio as a function of time for two cells (encircled in FIGS. 7B-7E) that responded to chloroquine but not histamine. Black bars indicate time of application of each indicated chemical. FIG. 7B: Fluorscence microscopic image of DRG cell labeled for GRP following calcium imaging. Chloroquine-sensitive cells were not labeled with GRP. FIG. 7C: SP. One chloroquine-sensitive cell (right) was lightly labeled. FIG. 7D: VGLUT2. One-chloroquine-responsive cell (left) was labeled for VGLUT2. FIG. 7E: Triple-staining (Merge). Two cells indicated by arrows were triple-labeled for GRP, SP and VGLUT2. FIG. 7F: Pie chart summarizing the percentages of chloroquine-responsive DRG cells (assessed by calcium imaging) that were co-labeled for SP, GRP and/or VGLUT2 (n=50). FIG. 7G: As in FIG. 7F, summarizing percentages of histamine-responsive DRG cells (assessed by calcium imaging) that were co-labeled for SP, GRP and/or VGLUT2 (n=40). Legend to the right of pie chart applies to both FIG. 7F and FIG. 7G. FIG. 7H: Incidence of DRG cells that responded to both chloroquine and histamine (assessed by calcium imaging) that were co-labeled for SP, GRP and/or VGLUT2 (n=14). FIG. 7I: As in FIG. 7H, for DRG cells that responded to chloroquine but not histamine (n=36). FIG. 7J: As in FIGS. 7H, 7I for DRG cells that responded to histamine but not chloroquine (n=26).

FIGS. 8A-8C Schematic diagram showing primary afferents and spinal dorsal horn neurons that transmit itch. FIG. 8A. Different pruriceptors release differing proportions of glutamate (Glu) and neuropeptides GRP or SP to excite NS and/or WDR neurons that signal itch. ●: GRP; ◯: SP; [symbol] : Glutamate. FIG. 8B: Pruriceptors release natriuretic polypeptide B (Nppb) and possibly glutamate to excite second-order spinal interneurons, which in turn release differing proportions of GRP, SP and glutamate to excite itch-signaling NS and/or WDR neurons. See text for further explanation. ●: GRP; ◯: SP; [symbol] : Glutamate; [symbol] : Nppb. FIG. 8C. Schematic of excitatory and inhibitory spinal interneurons. Itch mediators excite pruriceptors that may release glutamate and/or neuropeptides such as Nppb, GRP or SP. Intrathecal CNQX inhibits glutamatergic transmission from pruriceptors and/or excitatory spinal interneurons to itch-signaling neurons. Nociceptors release glutamate and SP to excite inhibitory spinal interneurons which inhibit itch-signaling spinal neurons. Loss of VGLUT2 in nociceptive afferents leads to reduced excitation of the inhibitory interneurons to disinhibit itch. This effect is proposed to outweigh any reduction in input from primary afferent pruriceptors.

### DETAILED DESCRIPTION OF THE INVENTION

### I. Definitions

As used in this specification and the appended claims, the singular forms “a,” “an,” and “the” include plural reference unless the context clearly dictates otherwise.

The term “inhibiting” or “inhibition,” as used herein, refers to any detectable negative effect on a target biological process, such as cellular signal transduction, including nerve signal transmission. Typically, an inhibition is reflected in a decrease of at least 10%, 20%, 30%, 40%, or 50% in the target process (e.g., histamine dependent or independent signal transmission), when compared to a control.

The “subject” is defined herein to include animals such as mammals, including, but not limited to, primates (e.g., humans), cows, sheep, goats, horses, dogs, cats, rabbits, rats, mice and the like. In some embodiments, the subject is a human.

The terms “treat”, “treating”, “treatment” and grammatical variations thereof as used herein, includes partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, pallatively or remedially.

The term “administering” refers to oral administration, administration as a suppository, topical contact, intravenous, intraperitoneal, intrathecal, epidural, intramuscular, intralesional, intranasal or subcutaneous administration, or the implantation of a slow-release device e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The term “effective amount,” as used herein, refers to an amount that produces therapeutic effects for which a substance is administered. The effects include the prevention, correction, or inhibition of progression of the symptoms of a disease/condition and related complications to any detectable extent. The exact amount will depend on the nature of the therapeutic agent, the manner of administration, and the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

The term “itch” as used herein includes chronic itch and acute itch. Itch or itching is a tingling or irritation of the skin that induces a subject to scratch the affected area. Itching may occur all over the whole body or only in one location. Methods and compositions provided herein are useful for treatment of chronic itch, acute itch, or a combination thereof. Itch may be detected by scratching behavior, or transmission of an itch signal along a sensory neuron as detected by measuring calcium influx or electrophysiology. Itch may be histamine dependent, which may be artificially induced by exogenous or ectopic application of the compound histamine. Itch may also be histamine independent, which may be artificially induced by exogenous or ectopic application of non-histamine irritant compounds including but not limited to chloroquine.

The term “itch signal transmission” as used herein refers to the transmission of an itch stimulus signal via one or more sensory neurons. Itch signal transmission may be histamine dependent or histamine independent. Histamine independent signals may be elicited artificially by ectopic or exogenous application of non-histamine irritants including but not limited to the compound chloroquine in a location proximal to one or more sensory neurons. Histamine dependent signals may be elicited artificially by ectopic or exogenous application of the compound histamine in a location proximal to one or more sensory neurons. Histamine dependent and independent itch signal transmission may be elicited by a variety of natural processes known in the art. Itch signal transmission may be detected by a number of methods known in the art including measurement of calcium ion influx using a calcium sensitive dye, electrophysiology, e.g. using a tungsten microelectrode, or behavioral observation, e.g. observing scratching behavior.

Inhibition of itch signal transmission may thus be detected by, for example, observing a reduction in calcium ion influx, a reduction in chloroquine or histamine responsive nerve firing, or a reduction in scratching behavior, or any other methods known in the art. Typically, an inhibition of itch signal transmission is reflected in a decrease of at least 10%, 20%, 30%, 40%, 50%, 60%, 67%, 70%, 75%, 80%, 90%, 95%, 97.5%, 98%, 99%, or 100% in histamine dependent or independent itch signal transmission, or a combination thereof, when compared to a control.

Compounds that have the same molecular formula but differ in the nature or sequence of bonding of their atoms or the arrangement of their atoms in space are termed “isomers”. Isomers that differ in the arrangement of their atoms in space are termed “stereoisomers”. “Stereoisomer” and “stereoisomers” refer to compounds that exist in different stereoisomeric forms if they possess one or more asymmetric centers or a double bond with asymmetric substitution and, therefore, can be produced as individual stereoisomers or as mixtures. Stereoisomers include enantiomers and diastereomers. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”. Unless otherwise indicated, the description is intended to include individual stereoisomers as well as mixtures. The methods for the determination of stereochemistry and the separation of stereoisomers are well-known in the art (see discussion in Chapter 4 of ADVANCED ORGANIC CHEMISTRY, 4th edition J. March, John Wiley and Sons, New York, 1992) differ in the chirality of one or more stereocenters.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge, S. M. et al., “Pharmaceutical Salts,” Journal of Pharmaceutical Science, 66:1-19, 1977). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug ester form. “Prodrug” s of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent. Prodrugs are frequently, but not necessarily, pharmacologically inactive until converted into the active drug. Prodrugs are typically obtained by masking a functional group in the drug believed to be in part required for activity with a progroup (defined below) to form a promoiety which undergoes a transformation, such as cleavage, under the specified conditions of use to release the functional group, and hence the active drug. The cleavage of the promoiety may proceed spontaneously, such as by way of a hydrolysis reaction, or it may be catalyzed or induced by another agent, such as by an enzyme, by light, by acid or base, or by a change of or exposure to a physical or environmental parameter, such as a change of temperature. The agent may be endogenous to the conditions of use, such as an enzyme present in the cells to which the prodrug is administered or the acidic conditions of the stomach, or it may be supplied exogenously.

Certain compounds of the present invention can exist in unsolvated forms as well as solvated forms, including hydrated forms. “Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers, regioisomers and individual isomers (e.g., separate enantiomers) are all intended to be encompassed within the scope of the present invention. These isomers can be resolved or asymmetrically synthesized using conventional methods to render the isomers “optically pure”, i.e., substantially free of its other isomers. If, for instance, a particular enantiomer of a compound of the present invention is desired, it may be prepared by asymmetric synthesis, or by derivation with a chrial auxilliary, where the resulting diastereomeric mixture is separated and the auxilliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diasteromers thus formed by fractional crystallization or chromatographic means well known in the art, and subsequent recovery of the pure enantiomers.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

### II. Introduction

Itch is thought to be signaled by histamine-dependent and -independent pathways. The present invention further elucidates the roles for substance P (SP), gastrin-releasing peptide (GRP), and glutamate in the spinal neurotransmission of both types of itch. In behavioral studies, systemic co-administration of antagonists of the SP neurokinin-1 (NK-1) and AMPA glutamate receptors attenuated intradermal chloroquine-evoked scratching behavior to a significantly greater extent than either antagonist delivered separately. Intrathecal administration of an antagonist of the GRP, NK-1 or AMPA glutamate receptor each significantly attenuated chloroquine-evoked scratching behavior. Co-administration of the NK-1 and GRP receptor antagonists was more effective, and administration of all three antagonists almost completely abolished scratching. Intrathecal administration of the AMPA receptor antagonist alone significantly attenuated histamine-evoked scratching behavior. Additionally a double-label strategy was employed to investigate molecular markers of pruritogen-sensitive dorsal root ganglion (DRG) cells. Cultured DRG cells responsive to histamine and/or chloroquine, identified by calcium imaging, were then processed for co-expression of SP, GRP or vesicular glutamate transporter type 2 (VGLUT2) immunofluorescence. Eighty percent were immunopositive for VGLUT2, with 10-18% also immunopositive for SP or GRP. In vivo single-unit recordings were made from superficial dorsal horn neurons responsive to intradermal injection of chloroquine or histamine. Chloroquine-evoked activity was partially reduced by spinal application of the AMPA antagonist alone. Co-application of the NK-1 or GRP antagonist with the AMPA antagonist produced stronger inhibition, while application of all three antagonists inhibited firing to the greatest extent, indicating a synergistic effect. Histamine-evoked activity was almost completely suppressed by spinal application of the AMPA receptor antagonists alone. These results indicate that SP, GRP and glutamate each partially contributes to histamine-independent itch, while glutamate appears to be the primary neurotransmitter involved in histamine-evoked itch. The data support the concept of co-application of any combination of NK-1, GRP and AMPA receptor antagonists to treat itch, including chronic itch, and itch that is resistant to antihistamines.

### III. Discussion

In the present invention, a multidisciplinary approach was used to further investigate the roles of SP, GRP and glutamate in histamine-dependent and -independent types of itch. Behavioral experiments were used to determine if antagonists of the NK-1 receptor, GRP receptor, or aminomethylphosphoric acid (AMPA) subtype of ionotropic glutamate receptor, attenuated scratching evoked by chloroquine or histamine in mice. Cell imaging was used to determine the expression of SP, GRP and vesicular glutamate transporter type 2 (VGLUT2), which is an essential transporter for glutamate release in the majority of A and C nociceptors (Scherrer et al., 2010), in pruritogen-sensitive primary sensory neurons. Finally, the presented invention examined whether the responses of murine superficial dorsal horn neurons to chloroquine or histamine are inhibited by spinal application of antagonists of the NK-1, GRP, or AMPA receptor.

In one object of the present invention, non-histaminergic itch is mediated by the intraspinal release of a combination of glutamate and the neuropeptides SP and GRP from chloroquine-sensitive pruriceptors to activate itch-signaling spinal neurons. In contrast, histamine-mediated itch appears to depend largely or exclusively on the release of glutamate from histamine-sensitive pruriceptors. These conclusions are supported by three lines of evidence. (1) In behavioral studies, co-application of antagonists of AMPA and NK-1 receptors more strongly suppressed chloroquine-evoked scratching than administration of either one individually. (2) Using a combination of calcium imaging and triple immunofluorescence labeling of DRG cells, 10-18% of chloroquine- or histamine-sensitive neurons co-expressed substance P or GRP while about 80% co-expressed VGLUT2. (3) Using an electrophysiological approach, id chloroquine-evoked responses of spinal dorsal horn neurons were partially suppressed by antagonists of NK-1, GRP and glutamate receptors, while co-application of the NK-1 and AMPA antagonists produced stronger suppression and the combined application of all three antagonists produced the greatest suppression. In contrast, the AMPA receptor antagonist CNQX almost completely abolished histamine-evoked firing of dorsal horn neurons. These data strongly support the concept of co-administration of AMPA, NK-1 and GRP receptor antagonists for improved treatment of forms of chronic itch that are resistant to antihistamines.

In some cases, non-histaminergic itch may be better controlled by a combinatorial approach using antagonists of the three neurotransmitters, namely glutamate, substance P and GRP, that are thought to be released from pruriceptors to excite spinal itch-signaling pathways. Data provided herein indicates that a combination of all three antagonists produces much stronger suppression of itch signaling than any individual antagonist, or co-application of the NK-1 and AMPA antagonists. This finding provides evidence to support the use of co-administration of any combination of these antagonists to treat antihistamine-resistant types of chronic itch under conditions such as atopic dermatitis, psoriasis, and kidney or liver disease.

Without wishing to be bound by theory, histamine-evoked itch appears to rely more exclusively on glutamatergic neurotransmission, such that application of an AMPA receptor antagonist nearly abolishes itch signaling. Thus in one object of the present invention, methods and compositions are provided for the use of AMPA receptor antagonists to treat histamine-mediated types of itch such as urticaria.

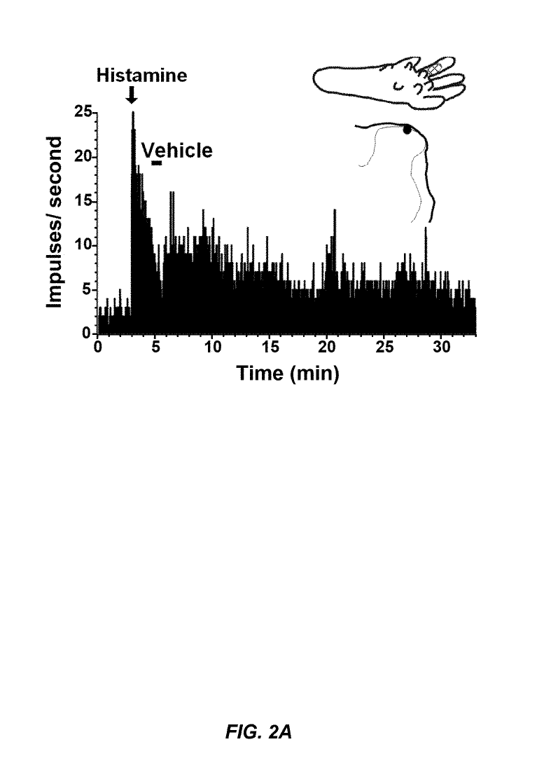

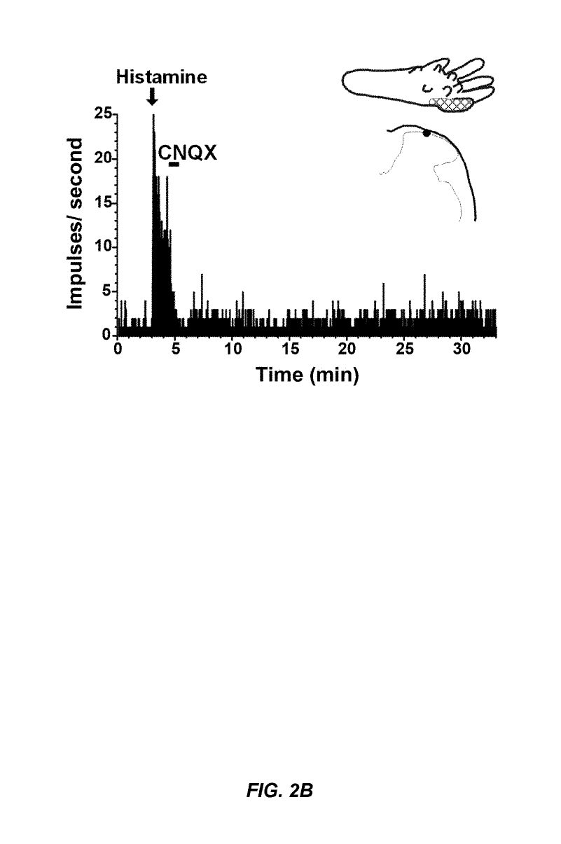

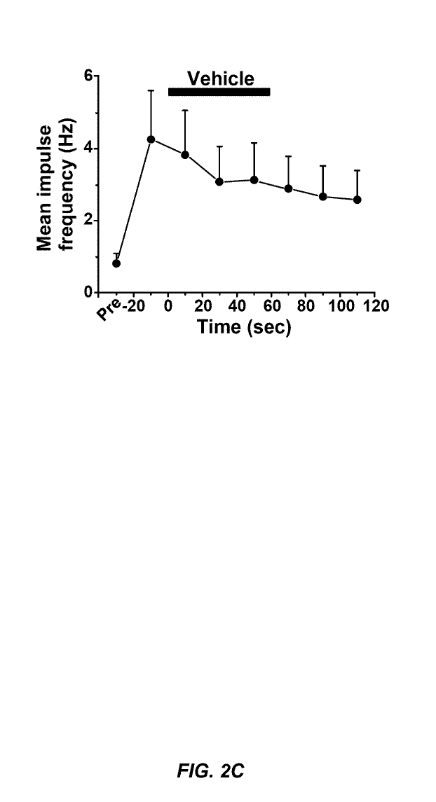

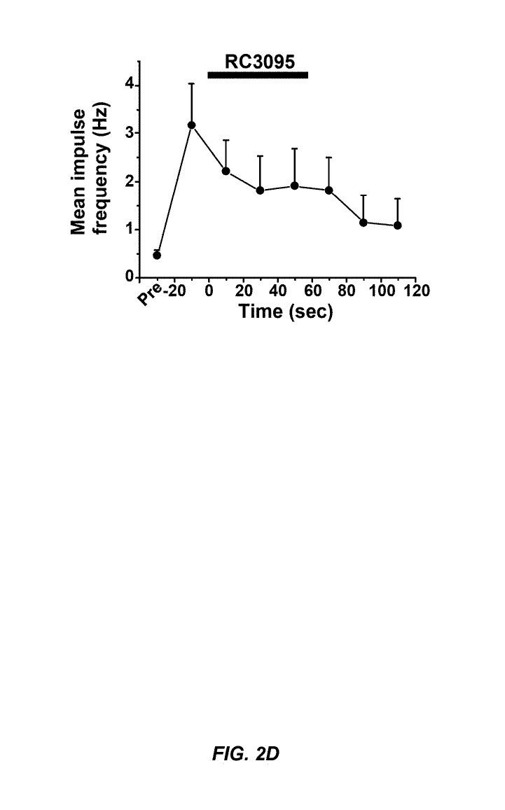

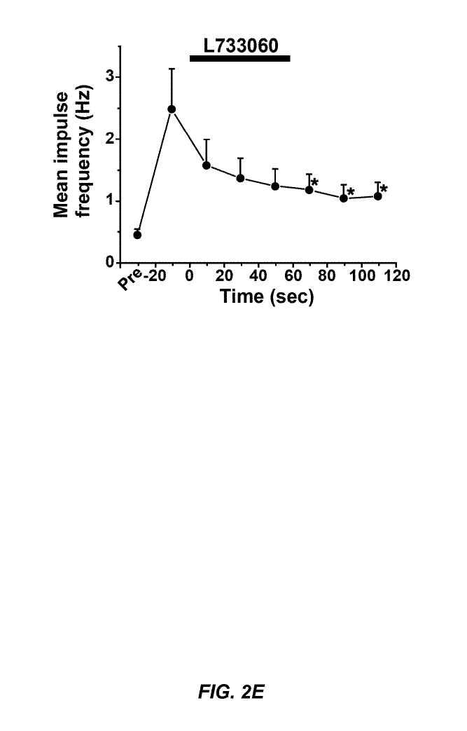

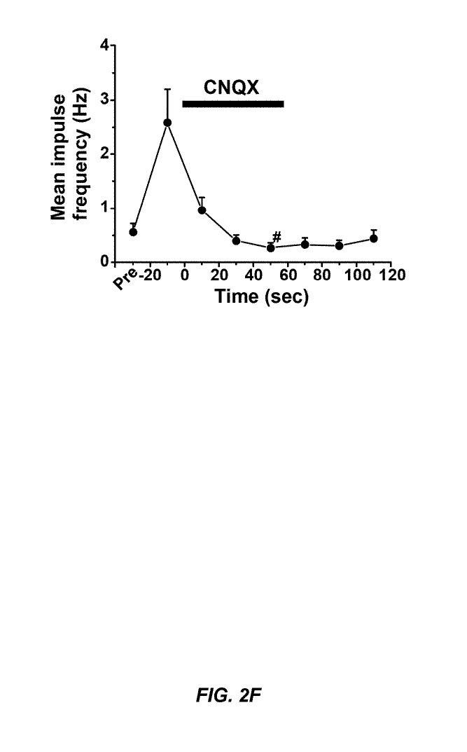

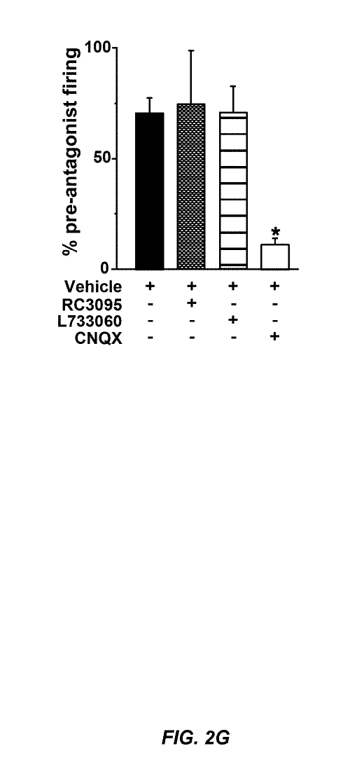

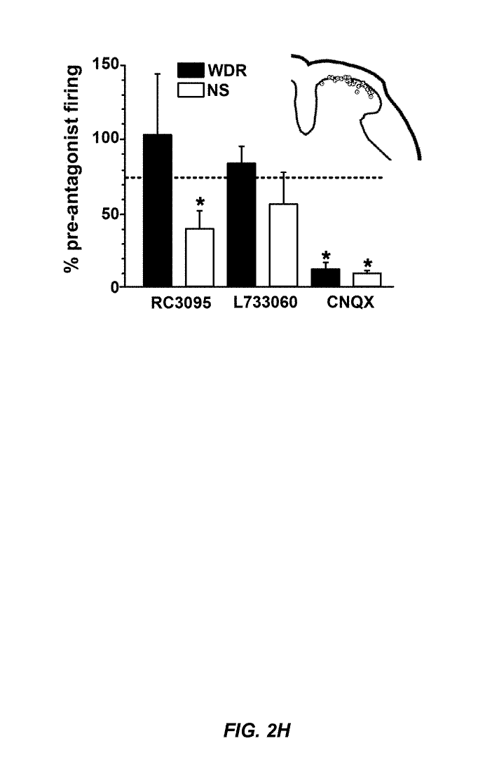

Behavioral studies. Systemic co-administration of the NK-1 and AMPA antagonists more effectively reduced chloroquine-evoked scratching behavior than either antagonist alone. Additional co-application of the GRP antagonist along with the other two did not improve the antipruritic efficacy. This might be due to a peripheral action of the GRP antagonist to induce scratching behavior (Andoh et al., 2011) which would counteract any central antipruritic action of the GRP antagonist. When administered intrathecally, the GRP antagonist by itself significantly reduced chloroquine-evoked scratching (FIG. 2A-H), indicating that it has a central antipruritic action that was additive or synergistic with the actions of the NK-1 and AMPA antagonist. Importantly, intrathecal co-injection of all three antagonists more effectively reduced chloroquine-evoked scratching than each individual antagonist or co-injection of the AMPA and NK-1 antagonists. Therefore, the present behavioral data strongly support the concept that systemic co-administration of an NK-1 and AMPA receptor antagonist more effectively reduces non-histaminergic chronic itch, and that intrathecal co-administration of NK-1, AMPA and GRP receptor antagonists provides the strongest antipruritic effect.

In contrast, intradermal histamine-evoked scratching behavior was not affected by individual intrathecal administration of either the NK-1 or the GRP receptor antagonist. Intrathecal administration of the AMPA receptor antagonist CNQX alone significantly reduced id histamine-evoked scratching behavior.

Cell imaging data. Nearly 20% of chloroquine-sensitive DRG cells co-expressed SP or GRP and 80% co-expressed VGLUT2, a marker for glutamatergic synapses. These data are fully consistent with combined roles for SP, GRP and glutamate in the spinal synaptic transmission of non-histaminergic itch signals. Slightly lower percentages of histamine-sensitive DRG cells co-expressed SP or GRP, while nearly 80% co-expressed VGLUT2. This is consistent with a role for glutamate in histamine-mediated itch, but does not rule out the possible contribution of SP and GRP as well.

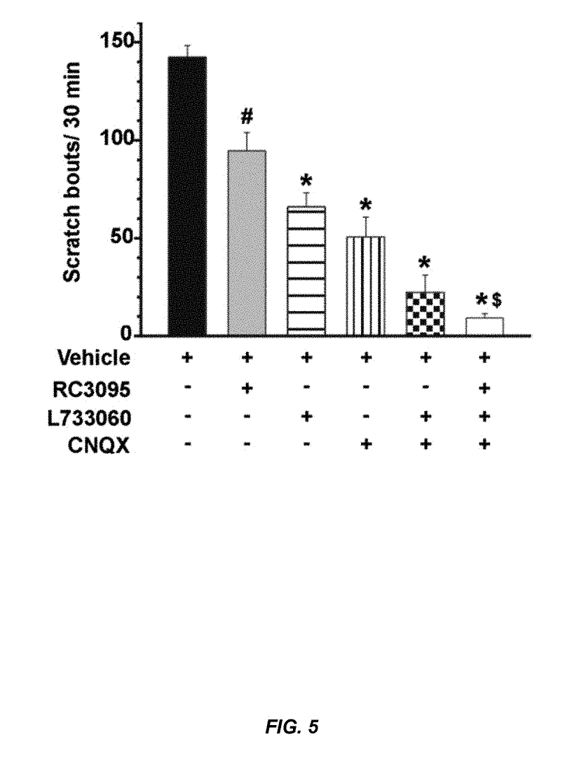

Spinal cord electrophysiology. A combination of antagonists more effectively reduces the spinal transmission of non-histaminergic itch signals. While the GRP and NK-1 receptor antagonists both reduced chloroquine-evoked firing (FIG. 5B, 5C), they did not produce a significant reduction in firing compared to vehicle superfusion (FIG. 5G). Only the AMPA receptor antagonist CNQX by itself significantly attenuated chloroquine-evoked firing (FIG. 5D, 5G). However, a combination of NK-1 and AMPA antagonists was more effective, and the triple combination of NK-1, GRP and AMPA antagonists was most effective. Thus, in one object of the present invention, methods and compositions are provided for co-administration of the three antagonists to relieve types of chronic itch that are resistant to antihistamines.

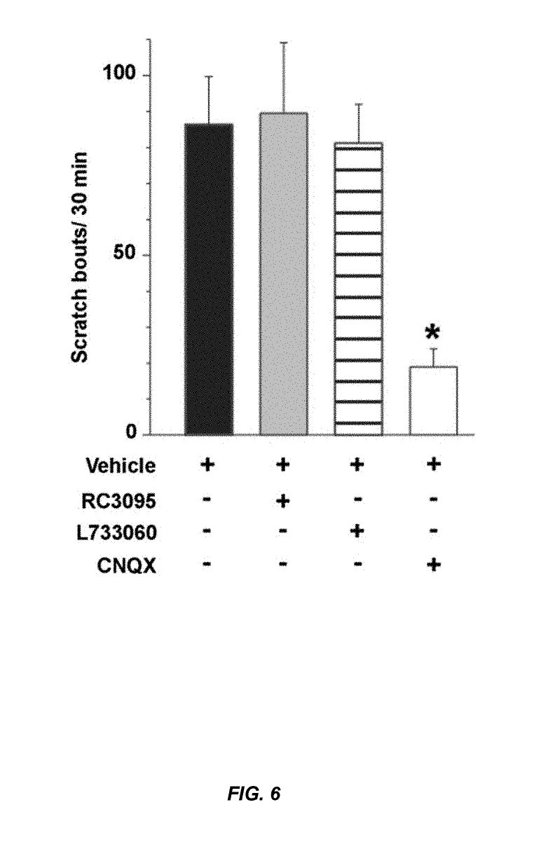

Glutamate also plays a role in the spinal transmission of histamine-mediated itch. For example, CNQX administered by itself almost completely abolished histamine-evoked firing of dorsal horn neurons (FIG. 6D, 6E).

Co-application of NK-1 and AMPA receptor antagonists, or intrathecal co-injection of NK-1, AMPA and GRP receptor antagonists, more effectively reduces non-histaminergic itch than any individual antagonist alone. For example, any of the antagonists listed in Table 1, or a combination thereof, could be utilized to inhibit itch. Additionally, glutamate is an important spinal neurotransmitter involved in histamine-mediated itch, which may be relieved by antagonists of the AMPA subtype of glutamate receptor. Additional gastrin releasing peptide receptor antagonists include, without limitation, those compounds disclosed in U.S. Pat. No. 5,047,502.

TABLE I

| Receptor | Drug name | Condition | Dose | Side Effects | Company | Reference |
|---|---|---|---|---|---|---|
| NK-1 | Aprepitant | Drug abuse, | 40-200 mg | generally mild. | | Green et al., J. |
| | | Aldosterone | p.o. | | | Urol. 176: |
| | | and Cortisol | | | | 2535-2540, |
| | | Secretion, | | | | 2006 |
| | | Multiple | | | | |
| | | Myeloma | | | | |
| NK-1 | AV608 | Irritable | 80-160 mg/ | well tolerated. | Avera | Tillish et al., |
| | | Bowel | day | (mild sinus | Pharmaceuticals | Aliment |
| | | Syndrome | po | congestion, | Industry | Pharmacol |
| | | (IBS), Social | | headache, | | Ther 35 360-367, |
| | | Phobia, | | nausea, | | 2012 |
| | | Overactive | | pharyngeal pain | | |
| | | Bladder | | and dizziness). | | |
| | | Syndrome | | | | |
| NK-1 | GR205171 | PTSD | 5 mg | well-tolerated | | Mathew et al., |
| | | | po | | | Europ |
| | | | | | | Neuropsycho- |
| | | | | | | pharmacol 21: |
| | | | | | | 221-229, 2011 |
| NK-1 | GW679769 | Nausea and | 50-150 mg | well tolerated, | Glaxo-Smith- | Singla et al., |
| | (casopitant) | Vomiting; | po | headache (mild | Klein | Anesthesiol |
| | | Fibro- | | dizziness, | | 113: 74-82, |
| | | myalgia, | | constipation) | | 2010. |
| | | Depression | | | | |
| NK-1 | LY686017 | Alcoholism | 1-100 mg | mild and | Eli Lilly and | Tauscher et al., |
| | | | po | transient | Co. | Europ |
| | | | | (somnolence | | Neuropsycho- |
| | | | | and dizziness) | | pharmacol (20, |
| | | | | | | 80-87, 2010. |
| NK-1 | L-759274 | Depression | 40 mg/ | well-tolerated. | | Kramer et al., |
| | | | day | | | Neuropsycho- |
| | | | po | | | pharmacol 29 |
| | | | | | | 385-392, 2004 |
| NK-1 | Orvepitant | PTSD, | 30-60 mg/ | | Glaxo-Smith- | http://clinicaltrialsfeeds.org/ |
| | (GW823296) | depression | day | | Klein | clinical-trials/show/NCT01000493 |
| NK-1 | vestipitant | tinnitus | 25 mg/ | | Glaxo-Smith- | http://clinicaltrialsfeeds.org/ |
| | | | day | | Klein | clinical-trials/show/NCT00394056 |
| NK-1 | GSK206136 | Depression, | 2-100 mg | | Glaxo-Smith- | http://clinicaltrialsfeeds.org/ |
| | | anxiety | po | | Klein | clinical-trials/show/NCT01059578 |
| NK-1 | GW67969 | Nausea, | | | Glaxo-Smith- | http://clinicaltrialsfeeds.org/ |
| | | vomiting | | | Klein | clinical-trials/show/NCT00108095 |
| NK-1 | GSK1144814 | schizophrenia | 100-200 mg | | Glaxo-Smith- | http://clinicaltrialsfeeds.org/ |
| | | | po | | Klein | clinical-trials/show/NCT01090440 |
| NK-1 | SSR240600C | Overactive | 500 mg | | | http://clinicaltrialsfeeds.org/ |
| | | Bladder | po | | | clinical-trials/show/NCT00174798 |
| NK-1 | Nolpitantium | Ulcerative | 600-1800 mg | | | http://clinicaltrialsfeeds.org/ |
| | Besylate | Colitis; | po | | | clinical-trials/show/NCT00232258 |
| | | Inflammatory | | | | |
| | | Bowel | | | | |
| | | Disease | | | | |
| AMPA | Talampanel | Epilepsy, | 25-75 mg, | Mild (fatigue, | | Iwamoto et al., |
| | | Parkinson's | 3x/day | dizziness, ataxia) | | Cancer 1776 |
| | | malignant | | | | 116: 1776 1782, |
| | | glioma | | | | 2010 |
| AMPA | Talampanel | ALS | 25-50 mg | | Teva | http://clinicaltrialsfeeds.org/ |
| | | | 3x/day | | Pharmaceutical | clinical-trials/show/NCT00696332 |
| | | | | | Industries | |
| AMPA | ZK 200775 | stroke | 0.6-6 mg/kg/hr | Sedation, | | Walters et al., |
| | | | iv | perception, | | Cerebrovasc Dis. |
| | | | | memory | | 20: 304-309, |
| | | | | | | 2005. |
| AMPA | GSK729327 | schizophrenia | 1-6 m | | Glaxo-Smith | |
| | | | | | Klein | |
| AMPA | topiramate | Parkinson's | | | | http://clinicaltrialsfeeds.org/ |
| | | disease | | | | clinicaltrials/show/NCT00296959 |
| AMPA | LY300164 | Parkinson's | | | | http://clinicaltrialsfeeds.org/ |
| | | disease, | | | | clinical-trials/show/NCT00004576 |
| | | dyskinesia | | | | |
| AMPA | BGG492 | tinnitus | | | Novartis | http://clinicaltrialsfeeds.org/ |
| | | | | | Pharmaceuticals | clinical-trials/show/NC701302873 |
| | | | | | Industry | |
| GRPR | RC-3095 | Breast | 8-96 ug/kg | hypergastrinemia | Zentaris | Schwartsmann et |
| | | cancer | sc | | | al., Invest New |
| | | | | | | Drugs 24: 403-412, |
| | | | | | | 2006 |

### IV. Pharmaceutical Compositions and Administration

The present invention provides pharmaceutical compositions or physiological compositions comprising an effective amount of a compound that inhibits itch signal transmission. For example, any of the compounds listed in Table 1 or a combination thereof are provided for inhibiting itch signal transmission. Compounds of the present invention include small chemicals, peptides, proteins, or natural products in both prophylactic and therapeutic applications. Such pharmaceutical or physiological compositions also include one or more pharmaceutically or physiologically acceptable excipients or carriers. Pharmaceutical compositions of the invention are suitable for use in a variety of drug delivery systems. Suitable formulations for use in the present invention are found in Remington's Pharmaceutical Sciences, Mack Publishing Company, Philadelphia, Pa., 17th ed. (1985). For a brief review of methods for drug delivery, see, Langer, Science 249: 1527-1533 (1990).

The pharmaceutical compositions of the present invention can be administered by various routes, e.g., oral, intradermal, subcutaneous, transdermal, intrathecal, epidural, intramuscular, intravenous, or intraperitoneal. Routes of administering the pharmaceutical compositions include local delivery to an organ or tissue suffering from a condition exacerbated by itch. For example, compositions may be delivered to the skin (e.g. intradermal or subcutaneous injection, or as a topical ointment) or one or more neurons responsible for transmitting an itch signal (e.g. epidural or intrathecal administration). Alternatively, compositions of the present invention may be administered systemically (e.g. oral or intravenous). Itch related skin conditions suitable for treatment by the methods and compositions of the present invention include uticaria, atopic dermatitis, contact dermatitis, dry skin, insect bites and stings, parasites such as pinworm, or lice, pityriasis rosea, rashes, seborrheic dermatitis, sunburn, folliculitis, impetigo, psoriasis, and post-burn injury.

Additionally, a variety of systemic conditions may result in chronic or acute itch symptoms treatable by the methods and compositions of the present invention. For example, kidney or liver diseases, cancers (e.g. lymphoma), blood disorders (e.g. polycythemia vera, leukemia), allergic reactions, iron deficiency, pregnancy, or reactions to medications, antibiotics (e.g. penicillin or sulfonamides), gold, griseofulvin, isoniazid, opiates, phenothiazines, or vitamin A. In some cases, such symptoms may be treated by delivery of compositions of the present invention to the skin (e.g. intradermal or subcutaneous injection, or as a topical ointment) or one or more neurons (e.g. epidural or intrathecal administration) responsible for transmitting an itch signal. Alternatively, compositions of the present invention may be administered systemically (e.g. oral or intravenous) to treat itch resulting from a systemic disease or condition.

For preparing pharmaceutical compositions containing an itch signal transduction inhibitor, inert and pharmaceutically acceptable carriers are used. The pharmaceutical carrier can be either solid or liquid. Solid form preparations include, for example, powders, tablets, dispersible granules, capsules, cachets, and suppositories. A solid carrier can be one or more substances that can also act as diluents, flavoring agents, solubilizers, lubricants, suspending agents, binders, or tablet disintegrating agents; it can also be an encapsulating material.

In powders, the carrier is generally a finely divided solid that is in a mixture with the finely divided active component, e.g., an AMPA, NK-1, or GRP receptor antagonist, or a combination thereof. In tablets, the active ingredient (e.g. an AMPA, NK-1, or GRP receptor antagonist or a combination thereof) is mixed with the carrier having the necessary binding properties in suitable proportions and compacted in the shape and size desired.

For preparing pharmaceutical compositions in the form of suppositories, a low-melting wax such as a mixture of fatty acid glycerides and cocoa butter is first melted and the active ingredient is dispersed therein by, for example, stirring. The molten homogeneous mixture is then poured into convenient-sized molds and allowed to cool and solidify.

Powders and tablets preferably contain between about 5% to about 70% by weight of the active ingredient of an inhibitor of histamine independent or dependent itch signal transmission. Suitable carriers include, for example, magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The pharmaceutical compositions can include the formulation of the active compound of an itch signal transmission inhibitor with encapsulating material as a carrier providing a capsule in which the modulator (with or without other carriers) is surrounded by the carrier, such that the carrier is thus in association with the compound. In a similar manner, cachets can also be included. Tablets, powders, cachets, and capsules can be used as solid dosage forms suitable for oral administration.

Liquid pharmaceutical compositions include, for example, solutions suitable for oral, topical, or parenteral administration, and suspensions, and emulsions suitable for oral, topical, or parenteral administration. Sterile water solutions of the active component (e.g., one or more histamine dependent and independent itch signal transmission inhibitors), or sterile solutions of the active component in solvents comprising water, buffered water, saline, PBS, ethanol, or propylene glycol are examples of liquid compositions suitable for parenteral administration. The compositions may contain pharmaceutically acceptable auxiliary substances as required to approximate physiological conditions, such as pH adjusting and buffering agents, tonicity adjusting agents, wetting agents, detergents, and the like.

Sterile solutions can be prepared by dissolving the active component (e.g., one or more histamine dependent and independent itch signal transmission inhibitors) in the desired solvent system, and then passing the resulting solution through a membrane filter to sterilize it or, alternatively, by dissolving the sterile compound in a previously sterilized solvent under sterile conditions. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9, and most preferably from 7 to 8.

The pharmaceutical compositions containing an itch signal transmission inhibitor can be administered for prophylactic and/or therapeutic treatments. In therapeutic applications, compositions are administered to a patient already suffering from chronic or acute itch in an amount sufficient to prevent, cure, reverse, or at least partially slow or arrest the symptoms of the condition and its complications, such as scratching behavior. An amount adequate to accomplish this is defined as a “therapeutically effective dose.” Amounts effective for this use will depend on the severity of the disease or condition and the weight and general state of the patient, but generally range from about 0.1 mg to about 2,500 mg of the inhibitor per day for a 70 kg patient, with dosages of from about 2.5 mg to about 500 mg of the inhibitor per day for a 70 kg patient being more commonly used.

In prophylactic applications, pharmaceutical compositions containing an itch signal transmission inhibitor (e.g. an AMPA, NK-1, or GRP receptor antagonist or a combination thereof) are administered to a patient susceptible to or otherwise at risk of developing a chronic or acute itch, in an amount sufficient to delay or prevent the onset of the symptoms. Such an amount is defined to be a “prophylactically effective dose.” In this use, the precise amounts of the modulator again depend on the patient's state of health and weight, and the source of the itch stimulus, but generally range from about 0.1 mg to about 2,500 mg of the inhibitor for a 70 kg patient per day, more commonly from about 2.5 mg to about 500 mg for a 70 kg patient per day.

Single or multiple administrations of the compositions can be carried out with dose levels and pattern being selected by the treating physician. In any event, the pharmaceutical formulations should provide a quantity of modulator sufficient to effectively inhibit transmission of histamine dependent or independent itch signal transmission or a combination thereof, either therapeutically or prophylactically.

All patents, patent applications, and other publications, including GenBank Accession Numbers, cited in this application are incorporated by reference in the entirety for all purposes.

### V. Examples

The following example is provided by way of illustration only and not by way of limitation. Those of skill in the art will readily recognize a variety of non-critical parameters that could be changed or modified to yield essentially the same or similar results.

### Example 1

### Abstract

The roles of substance P (SP), gastrin-releasing peptide (GRP), and glutamate in the spinal neurotransmission of histamine-dependent and -independent itch were investigated. In anesthetized mice, responses of single superficial dorsal horn neurons to intradermal (id) injection of chloroquine were reduced by spinal application of the AMPA/kainate antagonist CNQX. Co-application of CNQX plus a neurokinin-1 (NK-1) antagonist produced stronger inhibition, while co-application of CNQX, NK-1 and GRP receptor (GRPR) antagonists completely inhibited firing. Nociceptive-specific and wide dynamic range-type neurons exhibited differential suppression by CNQX plus either the GRPR or NK-1 antagonist, respectively. Neuronal responses elicited by id histamine were abolished by CNQX alone. In behavioral studies, individual intrathecal administration of a GRPR, NK-1 or AMPA antagonist each significantly attenuated chloroquine-evoked scratching behavior. Co-administration of the NK-1 and AMPA antagonists was more effective, and administration of all three antagonists abolished scratching. Intrathecal administration of CNQX alone prevented histamine-evoked scratching behavior. We additionally employed a double-label strategy to investigate molecular markers of pruritogen-sensitive dorsal root ganglion (DRG) cells. DRG cells responsive to histamine and/or chloroquine, identified by calcium imaging, were then processed for co-expression of SP, GRP or vesicular glutamate transporter type 2 (VGLUT2) immunofluorescence. Subpopulations of chloroquine- and/or histamine-sensitive DRG cells were immunopositive for SP and/or GRP, with >80% immunopositive for VGLUT2. These results indicate that SP, GRP and glutamate each partially contributes to histamine-independent itch, while glutamate is a major neurotransmitter involved in histamine-evoked itch. This suggests that co-application of one or more of NK-1, GRP and AMPA receptor antagonists is beneficial in treating itch, including histamine independent itch, histamine dependent itch, and chronic itch.

### Introduction

Chronic itch is a burdensome clinical problem that decreases the quality of life [Weisshaar E et al., The British journal of dermatology 155(5):957-964 (2006)], yet the neural mechanisms of itch are still not fully understood. Recent studies have implicated histamine-dependent and histamine-independent pathways in transmitting itch. The histamine-independent itch pathway involves members of the family of over 50 Mas-related G-protein coupled receptors (Mrgprs), in particular MrgprAs, MrgprB4-5, MrgprC11 and MrgprD, which are restricted to small diameter dorsal root ganglion (DRG) neurons in mice [Dong X et al., Cell 106(5):619-632 (2001)]. Chloroquine and the bovine adrenal medulla peptide 8-22 (BAMS-22) elicited itch-related scratching through MrgprA3 and MrgprC11, respectively, in mice [Liu Q et al., Cell 139(7):1353-1365 (2009)], and both compounds elicit itch in humans [Abila B et al., African journal of medicine and medical sciences 23(2):139-142 (1994); Sikand P et al., The Journal of neuroscience: the official journal of the Society for Neuroscience 31(20):7563-7567 (2011)]. β-alanine elicited itch via MrgprD [Liu Q et al., The Journal of neuroscience: the official journal of the Society for Neuroscience 32(42):14532-14537 (2012)]. It was recently reported that MrgprA3-expressing primary sensory neurons play a predominant role in itch evoked by chloroquine and other pruritogens [Han L et al., Nature neuroscience 16(2):174-182 (2012)], implying that spinal neurons with input from such chloroquine-sensitive primary afferents selectively signal itch sensation.

Neurokinin-1 (NK-1) and gastrin releasing peptide (GRP) receptor (GRPR)-expressing spinal neurons are implicated in signaling itch [Carstens E E et al., Neuroreport 21(4):303-308 (2010); Sun Y G et al., Science 325(5947):1531-1534 (2009)]. Their respective ligands, substance P (SP) and GRP, are partially involved in the spinal transmission of itch signals [Akiyama T et al., Journal of neurophysiology 109(3):742-748 (2013); Sun Y G and Chen Z F, Nature 448(7154):700-703 (2007)]. The predominant excitatory neurotransmitter, glutamate, is believed to also contribute to itch. A recent electrophysiological study suggested that glutamate acts as a neurotransmitter at GRP-sensitive spinal neurons [Koga K et al., Molecular pain 7:47 (2011)]. In contrast, the genetic ablation of the vesicular glutamate transporter type 2 (VGLUT2), which is essential for glutamate release from the majority of A- and C-fiber nociceptors [Scherrer G et al., Proceedings of the National Academy of Sciences of the United States of America 107(51):22296-22301 (2010)], resulted in reduced nocifensive behavior and enhanced spontaneous and pruritogen-evoked scratching [Lagerstrom M C et al., Neuron 68(3):529-542 (2010); Liu Y et al., Neuron 68(3):543-556 (2010)]. Another study reported that natriuretic polypeptide B (Nppb) is the primary transmitter released by pruritogen-sensitive primary afferents in mice [Mishra S K and Hoon M A, Science 340:968-971 (2013)]. Nppb excites GRPR-expressing spinal interneurons that are essential in transmitting itch, but not pain, signals to higher centers [Mishra S K and Hoon M A, Science 340:968-971 (2013)].

In the present study a multidisciplinary approach to investigate the roles of SP, GRP and glutamate in the spinal transmission of itch was utilized. Electrophysiological experiments were conducted to determine whether chloroquine-evoked responses of superficial dorsal horn neurons are inhibited by spinal application of antagonists of NK-1, GRP and/or glutamate aminomethylphosphoric acid (AMPA)/kainate receptors.

Complementary behavioral experiments investigated if these receptor antagonists alone or in combination attenuated chloroquine- and histamine-evoked scratching. Using a combination of calcium imaging followed by immunohistochemistry, the expression of SP, GRP and VGLUT2 in pruritogen-sensitive primary sensory neurons was investigated.

### Materials and Methods

Electrophysiology.

Experiments were performed using 118 adult male C57BL/6 mice (18-33 g) under a protocol approved by the UC Davis Animal Care and Use Committee. The single-unit recording from the lumbar spinal cord was conducted as previously detailed [Akiyama T et al., Journal of neurophysiology 102(4):2176-2183 (2009); Akiyama T et al., The Journal of neuroscience: the official journal of the Society for Neuroscience 29(20):6691-6699 (2009)]. Anesthesia was induced by sodium pentobarbital (60 mg/kg ip) and maintained by supplemental injections (10-20 mg/kg/hr). A gravity-driven perfusion system allowed artificial cerebrospinal fluid (Krebs: 117 mM NaCl, 3.6 mM KCl, 2.5 mM CaCl₂, 1.2 mM MgCl₂, 1.2 mM NaH₂PO₄, 25 mM NaHCO₃ and 11 mM glucose which was equilibrated with 95% 02 and 5% CO₂ at 37° C.) to be superfused continually over the exposed lumbosacral spinal cord [Akiyama T et al., PloS one 6(7):e22665 (2011)]. A tungsten microelectrode recorded single-unit activity in the lumbar spinal cord. A chemical search strategy [Akiyama T et al., Journal of neurophysiology 102(4):2176-2183 2009; Akiyama T et al., The Journal of neuroscience: the official journal of the Society for Neuroscience 29(20):6691-6699 (2009)] was used to identify and isolate chloroquine-responsive units. Our search strategy was intended to maximize the chance of isolating a chloroquine-responsive neuron; it was assumed that such neurons either gave rise to ascending projections, or served as interneurons in segmental scratch-reflex circuitry, and no attempt was presently made to distinguish between these possibilities.

Briefly, a small (˜0.25 μl) intradermal (id) microinjection of chloroquine (100 μg/μl) was made in the ventral hindpaw and a spontaneously active unit in the superficial lumbar dorsal horn (depth<300 um) was isolated. After the spontaneous activity had waned, chloroquine (1 μl, 100 μg/μl) was injected again at the same site through the same needle. Only units exhibiting an increase of >30% in firing to this second test microinjection of chloroquine were selected for further study. Responses were usually recorded for at least 30 minutes, although in many cases unit firing declined over a shorter period.

During the time that the unit exhibited a relatively stable level of chloroquine-evoked firing (usually 1 minute post-injection), one of the following antagonists was successively delivered directly to the spinal cord for 1 min; the GRP receptor antagonist RC-3095 (20 μM), the NK-1 receptor antagonist L-733060 (200 μM), the AMPA/kainate receptor antagonist CNQX (100 μM), a combination of CNQX (100 μM) and L-733060 (200 μM), or a combination of RC-3095 (20 μM), CNQX (100 μM) and L-733060 (200 μM).

When unit firing to the second chloroquine injection declined and reached a steady level, the unit's mechanosensitive receptive field was determined. The perimeter of the mechanical receptive field was mapped using a von Frey filament (55 mN bending force) by determining sites at which the unit either did (within receptive field) or did not (outside receptive field) respond to at least 3 of 5 repeated applications. The rationale for choosing this bending force is that it was sufficient to map the maximum extent of the mechanical receptive field as assessed by comparison of receptive field sizes mapped using a range of von Frey stimuli (0.7 mN: 1.2±0.6 mm², 6.9 mN: 3.7±0.9 mm², 55 mN: 9.9±1.4 mm², 758 mN: 10.3±1.4 mm²). Units were classified as wide dynamic range (WDR)-type if they responded in a graded manner to innocuous mechanical stimulation (brushing, cotton wisp) and noxious pinch, or nociceptive-specific (NS) if they responded to noxious pinch (and to the 55 mN von Frey stimulus) but not to the cotton wisp or brush stimuli. The properties are similar to those of WDR and NS units shown in previous studies to respond to histamine, serotonin, the PAR-2/MrgprC11 agonist SLIGRL, or chloroquine [Akiyama T et al., Journal of neurophysiology 102(4):2176-2183 (2009); Akiyama T et al., The Journal of neuroscience: the official journal of the Society for Neuroscience 29(20):6691-6699 (2009)]. In some units, at least 5 minutes after the noxious pinch stimulus, either the NK-1 receptor antagonist, the AMPA/kainate receptor antagonist, or a mixture of both, was superfused directly over the spinal cord for 1 min. At the end of the antagonist superfusion, the noxious pinch stimulus was delivered again at the same site on the receptive field. Thirty minutes later, the noxious pinch stimulus was delivered in the same manner.

Following mechanical stimulation, histamine (50 μg) was injected id within the same receptive field at a different location via a separate injection cannula. Following the id histamine injection, we tested the effects of antagonists for the receptors of NK1, GRP, or AMPA/kainate in the same manner as described above for chloroquine. Units were then tested with topical hindpaw application of allyl isothiocyanate (AITC; mustard oil, Sigma; 75% in mineral oil, 2 μl). Following the AITC application, we tested the effects of the NK-1 receptor antagonist, the AMPA/kainate receptor antagonist, or a combination of both antagonists in the same manner as described above for chloroquine.

Action potentials were recorded to a computer and counted using Chart software (AD Instruments, Colorado Springs Colo.) and Spike2 software (CED Instruments). Ongoing responses elicited by chloroquine, histamine, or AITC were averaged at 20-second intervals before, during and after the antagonist application, and compared by one way repeated-measures analysis of variance (ANOVA) followed by post-hoc Bonferroni test, with p<0.05 set as significant. The mean firing rate was calculated over a 20-second period 40 seconds after the antagonist application, and compared by one way ANOVA followed by post-hoc Bonferroni test, with p<0.05 set as significant. The criterion for decrease in ongoing firing was >70% decrease below the ongoing activity elicited by pruritogen over a 20-second period 40 seconds after the antagonist application. Mean peak responses elicited by noxious pinch were compared by one way repeated-measures ANOVA followed by post-hoc Bonferroni test, with p<0.05 set as significant. At the end of each experiment, an electrolytic lesion was made at the spinal cord recording site. The spinal cord was postfixed in 10% buffered formalin, cut in 50 μm frozen sections, and examined under the light microscope to identify lesions.

Behavior.

Experiments were conducted using adult male C57BL/6 mice (Simonsen, Gilroy, Calif.; 19-25 g) under a protocol approved by the UC Davis Animal Care and Use Committee. The fur on the rostral back was shaved and mice were habituated to the Plexiglas recording arena one week prior to testing. For intrathecal injections, either vehicle (saline), the GRP receptor antagonist RC-3095 (0.3 nmol; Sigma-Aldrich, St. Louis Mo.), the NK-1 antagonist L-733060 (22.7 nmol; Tocris Bioscience, Minneapolis, Minn.), the AMPA/kainate antagonist CNQX (20 nmol; Tocris Bioscience), a combination of CNQX (20 nmol) and L-733060 (22.7 nmol), or a combination of RC-3095 (0.3 nmol), CNQX (20 nmol) and L-733060 (22.7 nmol), was administered by lumbar puncture, followed 5 minutes later by id injection (10 μl) of either chloroquine (193 nmol; Sigma-Aldrich) or histamine (271 nmol; Sigma-Aldrich). Microinjections were made id in the nape of the neck using a 30 G needle attached to a Hamilton microsyringe by PE-50 tubing. Immediately after the id injection the mouse was placed into the arena and videotaped from above for 30 min. Generally 3-4 mice were injected and videotaped simultaneously. Immediately after commencing videotaping all investigators left the room.

Videotapes were reviewed by investigators blinded to the treatment, and the number of scratch bouts was counted at 5-minute intervals. A scratch bout was defined as one or more rapid back-and-forth hind paw motions directed toward and contacting the injection site, and ending with licking or biting of the toes and/or placement of the hind paw on the floor. Hind paw movements directed away from the injection site (e.g., ear-scratching) and grooming movements were not counted. One-way ANOVA followed by the Bonferroni post-test was used to compare the total number of scratch bouts across pretreatment groups. In all cases p<0.05 was considered to be significant. Data for effects of individual antagonist of either NK1 or GRP receptor on scratching evoked by either histamine or chloroquine were modified from our recent study [Akiyama T et al., Journal of neurophysiology 109(3):742-748 (2013)].

Calcium Imaging.

A total of 20 adult male C57BL/6 mice (Simonsen, Gilroy, Calif.; 7-9 weeks old, 18-21 g) was used under a protocol approved by the UC Davis Institutional Animal Care and Use Committee. The animal was euthanized under sodium pentobarbital anesthesia and lumbar DRGs were acutely dissected and enzymatically digested at 37° C. for 10 minutes in Hanks's balanced salt solution (HBSS; Invitrogen, Carlsbad, Calif.) containing 20 units/ml papain (Worthington Biochemical, Lakewood, N.J.) and 6.7 mg/ml L-cysteine (Sigma), followed by 10 minutes at 37° C. in HBSS containing 3 mg/ml collagenase (Worthington Biochemical). The ganglia were then mechanically triturated using fire-polished glass pipettes. DRG cells were pelleted, suspended in MEM Eagle's with Earle's BSS (Gibco) containing 100 U/ml penicillin, 100 μg/ml streptomycin (Gibco), 1× vitamin (Gibco) and 10% horse serum (Quad Five, Ryegate, Mont.), plated on poly-D-lysine-coated glass coverslips, and cultured for 16-24 hr.

DRG cells were incubated in Ringers solution (pH7.4; 140 mM NaCl, 4 mM KCl, 2 mM CaCl₂, 1 mM MgCl₂, 10 mM HEPES and 4.54 mM NaOH) with 10 μM of Fura-2 AM and 0.05% of Pluronic F-127 (Invitrogen). Coverslips were mounted on a custom made aluminum perfusion block and viewed through an inverted microscope (Nikon TS100, Technical Instruments, San Francisco Calif.). Fluorescence was excited by UV light at 340 nm and 380 nm alternately and emitted light was collected via a CoolSnap camera attached to a Lambda LS lamp and a Lambda optical filter changer (Sutter Instrument Company, Novato, Calif.). Ratiometric measurements were made using Simple PCI software (Compix Inc, Cranberry Township, Pa.) every 3 sec.

Solutions were delivered by a solenoid-controlled 8-channel perfusion system (ValveLink, AutoM8). Chloroquine (300 μM) or histamine (100 μM) was delivered, followed by potassium at a concentration of 144 mM. Stimulus duration was 30 sec. Ratios were normalized to baseline. Cells were judged to be sensitive if the ratio value increased by more than 10% of the resting level following chemical application. Only cells responsive to high-K+ were included for analysis. After the experiment, coverslips were marked with a diamond pen to provide landmarks for alignment with subsequent immunohistofluoresence labeling of the same cells.

Immunocytochemistry.

After calcium imaging, DRG cells in the culture dish were fixed in 4% paraformaldehyde followed by 30% sucrose and then incubated with 5% normal serum. They were immunostained with anti-rabbit GRP antibody (1:500; ImmunoStar Inc, Hudson, Wis.), anti-rat SP antibody (1:500; Millpore, Billerica, Mass.) and anti-guinea pig VGLUT2 antibody (1:300; Frontier Institute Co Ltd, Japan) at 4° C. overnight, followed by incubation with the corresponding secondary antibody conjugated with Alexa Fluor 350 (1:300; Life Technologies Inc, Grand Island, N.Y.), Alexa Fluor 488 (1:500; Life Technologies Inc) and Alexa Fluor 594 (1:500; Life Technologies Inc) for 2 hours. Images were captured using a fluorescence microscope (Nikon Eclipse Ti; Technical Instruments, San Francisco Calif.). Immunohistofluorescent images were aligned with images captured during calcium imaging to determine the percentages of pruritogen-responsive DRG cells that were triple-labeled for SP, GRP and VGLUT2.

### Results

### Electrophysiology

Chloroquine-Evoked Responses were Inhibited More by Antagonist Co-Application.

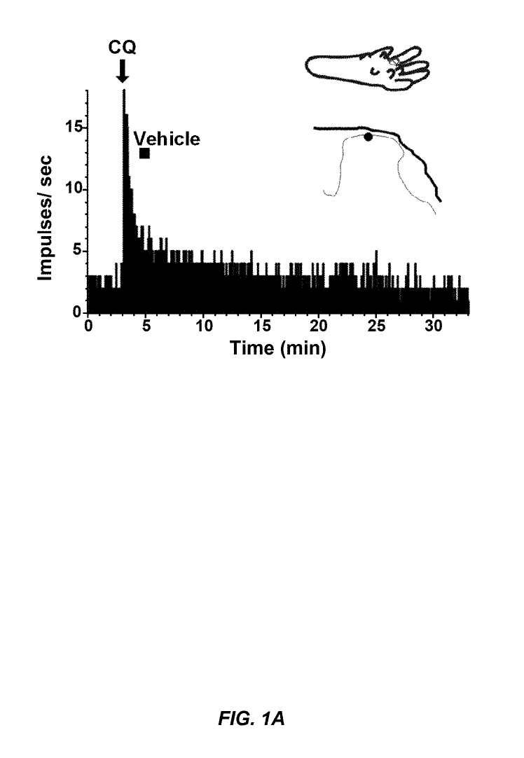

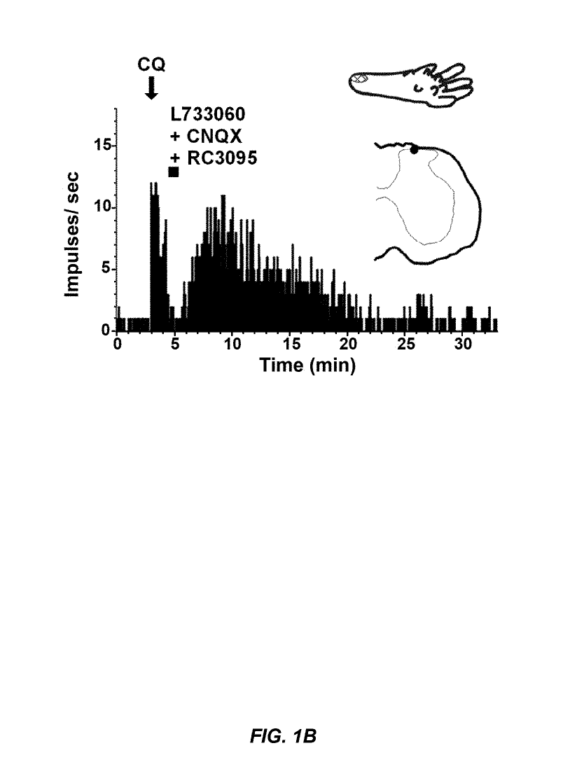

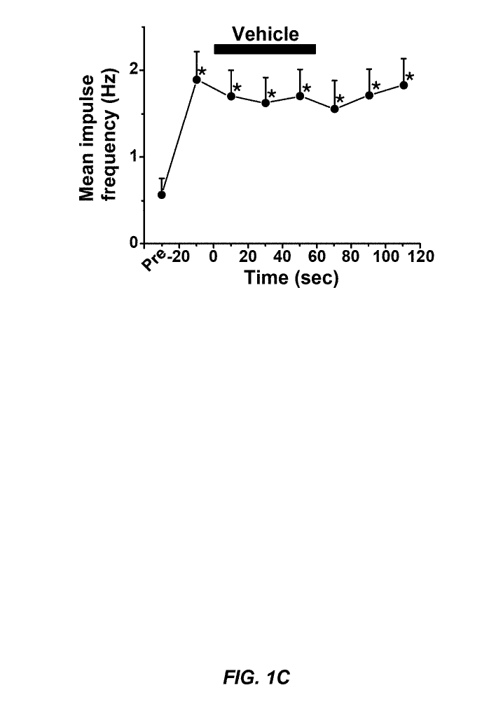

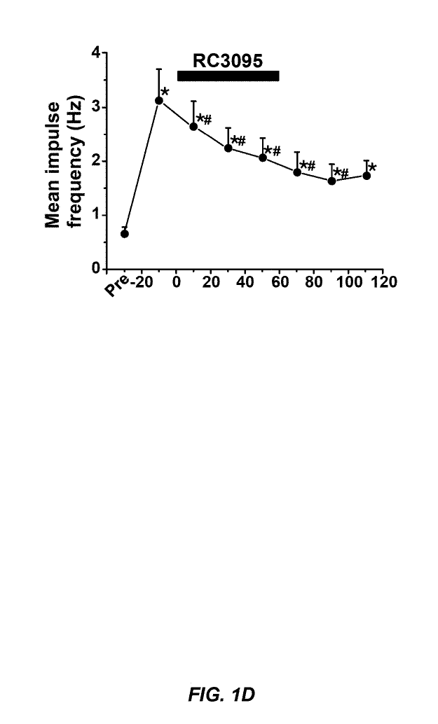

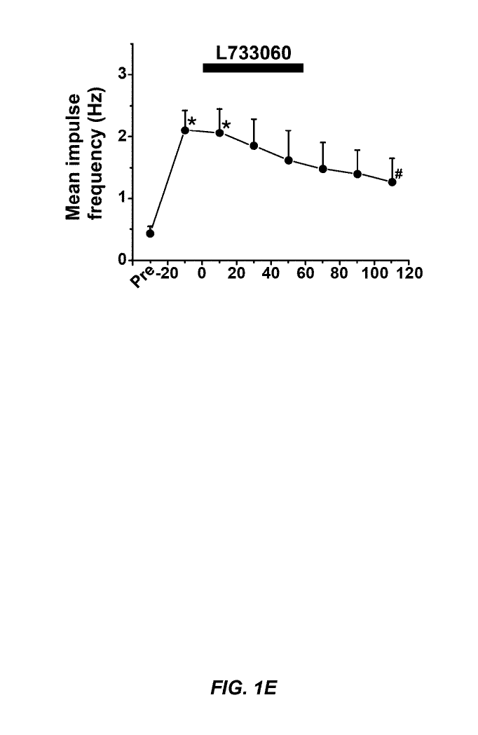

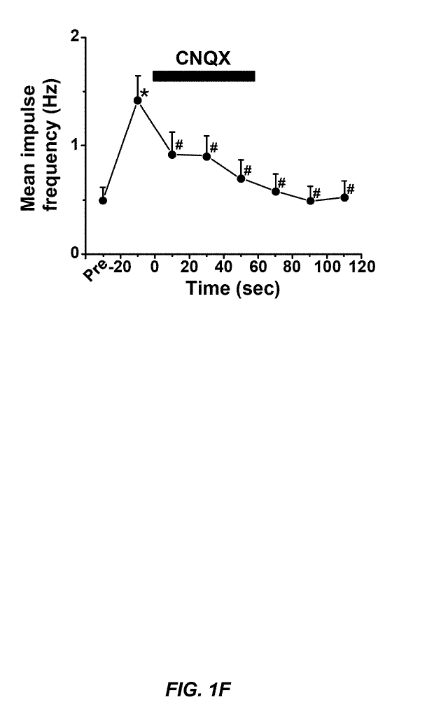

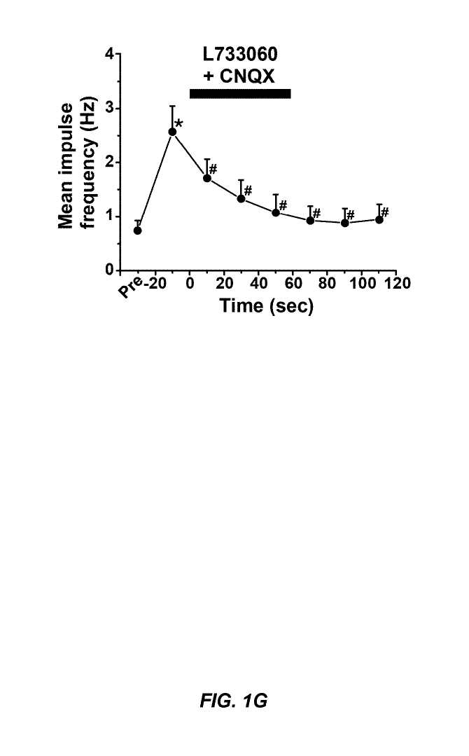

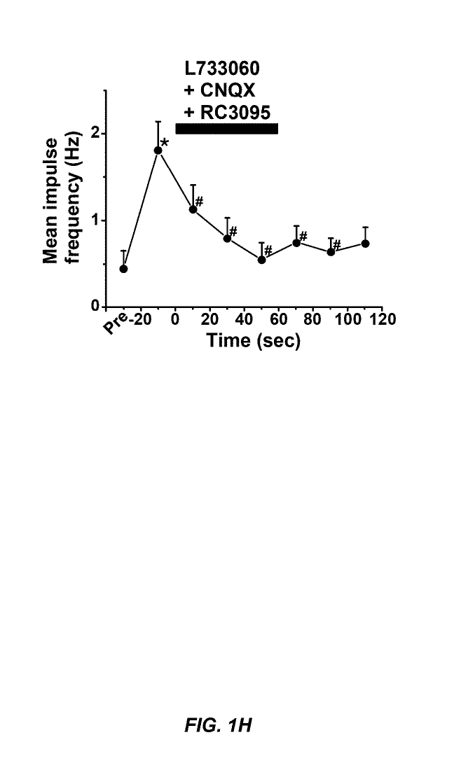

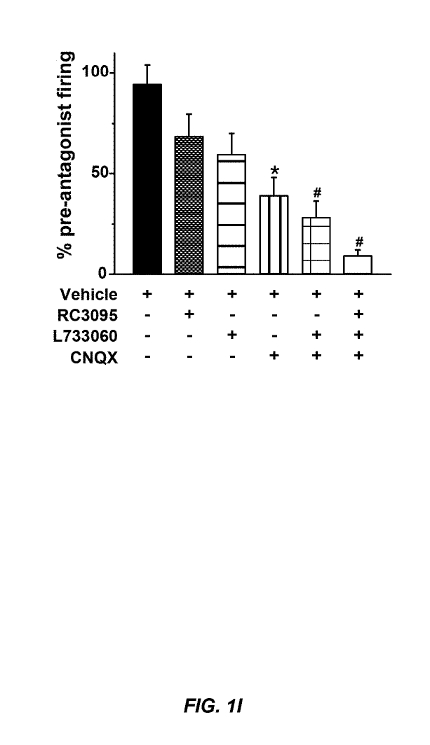

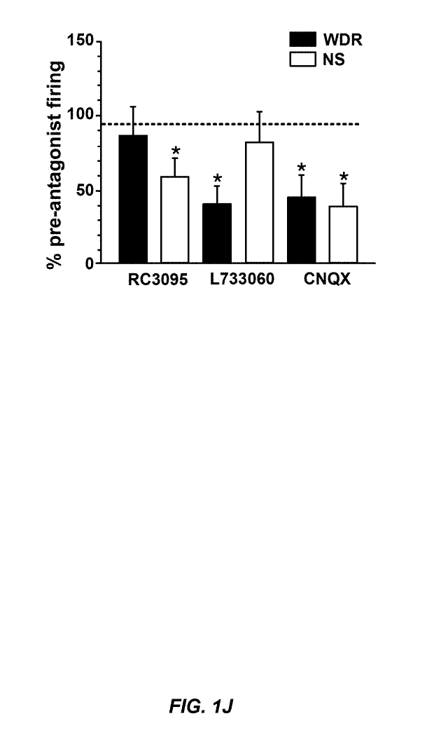

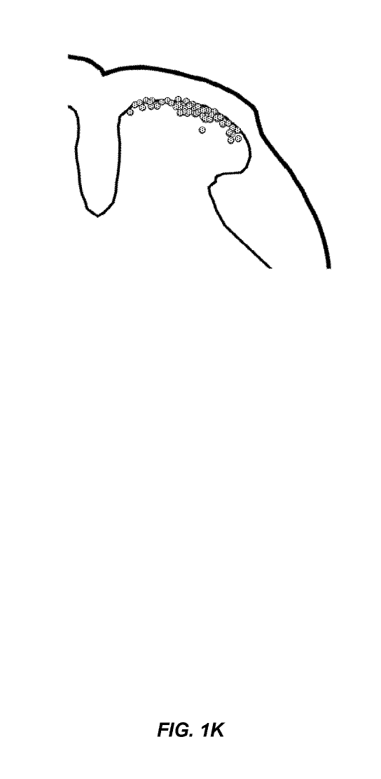

A total of 210 chloroquine-responsive superficial dorsal horn neurons (78 NS, 73 WDR, 59 uncharacterized) was tested with spinal application of antagonists. The units were located in the superficial dorsal horn at a mean depth of 80.0 μm+/−4.8 (SEM) below the surface (FIG. 1K). All units had mechanosensitive receptive fields on the ipsilateral hind paw, and responded to id microinjection of chloroquine within the receptive field. FIG. 1A shows an example of the prolonged response of a superficial dorsal horn unit to id chloroquine. The graphs in FIG. 1C-H plot averaged neuronal responses, quantified as the mean firing rate averaged over the preceding 20-second period. Neuronal activity increased immediately following id injection of chloroquine to a level that was significantly greater than the pre-injection baseline (FIG. 1A-H). The mean chloroquine-evoked response was also significantly greater than saline (vehicle)-evoked responses in all treatment groups (C: vehicle group, p=0.004 vs. pre; D: GRPR antagonist, p=0.0003; E: NK-1 antagonist, p=0.001; F: CNQX, p=0.026; G: NK-1+AMPA antagonist, p=0.0007; H: all 3 antagonists, p=0.007; unpaired t-test).

To investigate the role of SP, GRP and glutamate as neurotransmitters that excite chloroquine-sensitive spinal neurons, one or more antagonists of these neurotransmitters was superfused over the spinal cord during the chloroquine-evoked response. In FIG. 1C, mean responses are aligned with the onset of vehicle superfusion (black bar) at time 0. Chloroquine-evoked firing usually peaked within the first few seconds post-injection and continued over the ensuing 120 seconds, allowing us to test the effect of antagonist superfusion during this period of activity. As a control, vehicle was superfused and shown to have no effect on chloroquine-evoked firing. FIG. 1A shows an individual example, and FIG. 1C shows that the chloroquine-evoked firing rate remained significantly above baseline (FIG. 1C, *) during and after spinal superfusion of vehicle. Most units exhibited little or no adaptation in firing rate during the 60-second period of vehicle superfusion, while one unit exhibited a decline of nearly 70%. Using this as a conservative criterion, 0/23 units tested exhibited a decline by more than 70% in the chloroquine-evoked firing rate relative to that observed prior to the spinal superfusion (Table 2).

TABLE 2
Percentages of dorsal horn units that exhibited a decrease of 70% or more
in firing rate following spinal superfusion of antagonists.

| | | | | | | RC3095 + |
|---|---|---|---|---|---|---|
| | | | | | L733060 + | L733060 + |
| | VH | RC3095 | L733060 | CNQX | CNQX | CNQX |
| Chloroquine | 0% (0/23) | 24% (7/29) | 30% (9/30) | 58% (18/31) | 71% (25/35) | 85% (24/28) |
| Histamine | 25% (4/16) | 55% (10/18) | 33% (6/18) | 100% (14/14) | | |
| AITC | 27% (7/26) | | 22% (2/9) | 62% (8/15) | 89% (17/19) | |

Superfusion of the GRP receptor antagonist RC3095 resulted in an overall decline in mean firing rate (FIG. 1D). During spinal superfusion of RC3095, mean responses were still above pre-chloroquine baseline levels, but decreased significantly relative to the chloroquine-evoked response prior to spinal superfusion (FIG. 1D, #), indicating that the GRP receptor antagonist attenuated ongoing activity elicited by chloroquine. Twenty-four percent of units exhibited a decline of 70% or more during superfusion with the GRP antagonist (Table 2).

Chloroquine-evoked firing was also significantly attenuated after the cessation of superfusion with the NK-1 receptor antagonist L733060 (FIG. 1E, #), with 30% of units exhibiting a decline of 70% or more. Chloroquine-evoked firing was also significantly attenuated during and after spinal superfusion with the AMPA/kainate receptor antagonist CNQX (FIG. 1F), with 58% declining by 70% or more. Importantly, combinations of the NK-1 and AMPA/kainate receptor antagonist (FIG. 1G), or all three antagonists (FIG. 1B, 1H), significantly attenuated chloroquine-evoked firing during and after their spinal superfusion with 71% and 85% exhibiting reductions in firing rate of 70% or greater, respectively (Table 2). FIG. 1B shows an example in which spinal superfusion with all three antagonists completely suppressed chloroquine-evoked firing, followed by recovery of firing.

FIG. 1I summarizes the suppression of id chloroquine-evoked neuronal firing by antagonists. Chloroquine-evoked firing during the 40-60-second period after onset of spinal superfusion was normalized to the firing rate 20 seconds prior to the superfusion, and these normalized values were compared with the firing rate 40-60 seconds after superfusion with vehicle. By this analysis, chloroquine-evoked firing was increasingly reduced by the GRPR, NK-1 and AMPA/kainate receptor antagonists applied individually, more strongly by the combination of NK-1 and AMPA/kainate antagonists, and most strongly by co-application all three antagonists.

We additionally determined if antagonists differentially affected dorsal horn neurons based on their subclassification as WDR or NS. FIG. 1J shows effects of the three antagonists on chloroquine-evoked responses of WDR (n=36) and NS units (n=37). The GRPR antagonist significantly reduced chloroquine-evoked firing in NS but not WDR cells. In contrast, the NK-1 antagonist significantly reduced chloroquine-evoked firing in WDR but not NS cells. CNQX significantly reduced chloroquine-evoked firing in both WDR and NS cells. It should be noted that the effects of antagonists were not related to whether the units additionally responded to histamine or not. Of the 75 chloroquine-responsive units that also responded to histamine, 42 were classified as NS cells and 33 as WDR. Of the units that responded to chloroquine but not histamine, 5 were NS and 5 were WDR.

CNQX Alone Inhibited Histamine-Evoked Responses.

The large majority of chloroquine-responsive units (75/85) also responded to id injection of histamine. Following id histamine, unit firing increased abruptly; an example is shown in FIG. 2A. The graphs in FIGS. 2C-2F plot averaged neuronal responses to histamine, quantified as the mean firing rate averaged over the preceding 20-second period. In each instance, neuronal activity increased immediately following id injection of histamine to a level that was significantly greater than the vehicle (saline)-evoked response (C: vehicle, p=0.018 vs. pre; D: GRPR antagonist, p=0.011; E: NK-1 antagonist, p=0.015; F: CNQX, p=0.009; unpaired t-test).

The individual example in FIG. 2B shows that spinal superfusion with CNQX completely suppressed histamine-evoked firing, as confirmed for the unit population (FIG. 2F) in which superfusion with CNQX significantly reduced the mean firing rate to the pre-histamine baseline. Overall, CNQX significantly attenuated histamine-evoked firing to a level not different from that elicited by id injection of vehicle (saline) (FIG. 2G, white bar), with 100% of units exhibiting a reduction in firing of more than 70% (Table 2). Both the GRPR and NK-1 antagonists numerically reduced histamine-evoked firing compared to vehicle (FIGS. 2D, 2E, 2G), with 55% and 33% being reduced by more than 70%, respectively (Table 2). The overall effects, however, were not significantly different from vehicle (FIG. 2C).

FIG. 2H shows effects of the three antagonists on histamine-evoked responses of WDR (n=26) and NS units (n=21). Both NS and WDR unit responses to histamine were significantly reduced by CNQX (FIG. 2H). The GRP antagonist significantly reduced the mean histamine-evoked response of NS but not WDR units (FIG. 2H). The NK1 antagonist failed to reduce histamine-evoked firing in either WDR or NS units.

Lack of Effect of Antagonists on Baseline Activity.

There was no significant effect of any of the antagonists on the baseline activity of superficial dorsal horn neurons that were subsequently show to respond to pruritogens. We compared the neuronal firing rate averaged over the 1-minute period prior to antagonist application with that during a comparable 1-minute period following application of the antagonist (before application of pruritic or noxious stimuli). Values prior to, and after administration of each antagonist individually or in combination, are as follows. NK-1 antagonist L-733060: pre-application 0.88 Hz±0.23 (SEM), post-antagonist 0.72 Hz±0.19 (n=10). GRPR antagonist RC-3095: pre-application 0.54 Hz±0.54, post-antagonist: 0.53 Hz±0.53 (n=4). CNQX: pre-application 0.53 Hz±0.14, post-antagonist 0.64 Hz±0.21 (n=22). CNQX+L-733060: pre-application 0.52 Hz±0.17, post-antagonists 0.43 Hz±0.11 (n=26). CNQX+L-733060+RC-3095: pre-application 0.14 Hz±0.06, post-antagonists 0.17 Hz±0.06 (n=9).

CNQX Reduced Firing Elicited by AITC and Noxious Pinch.

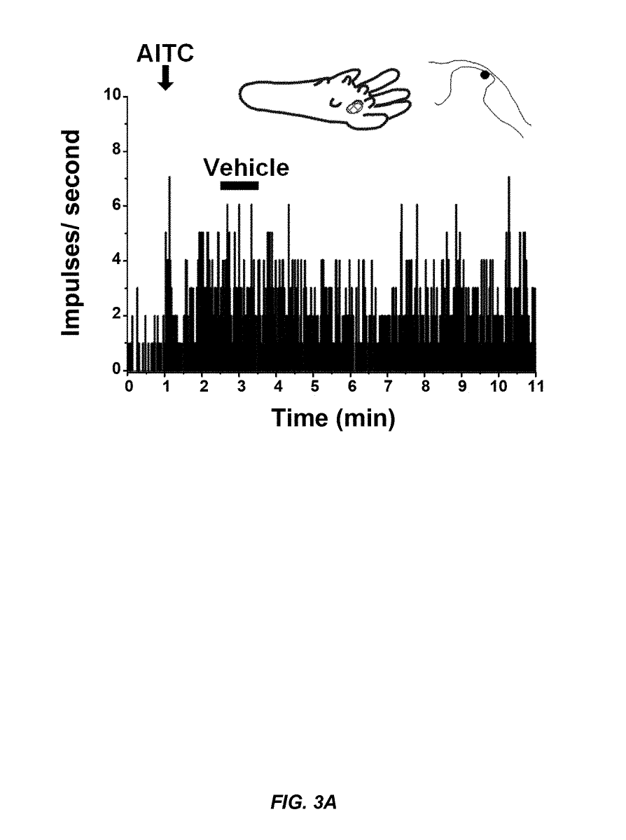

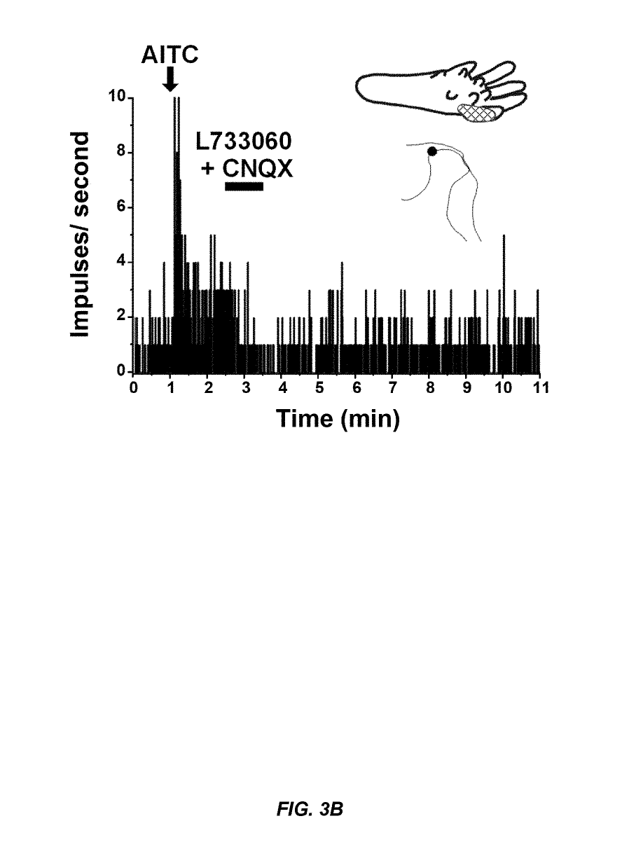

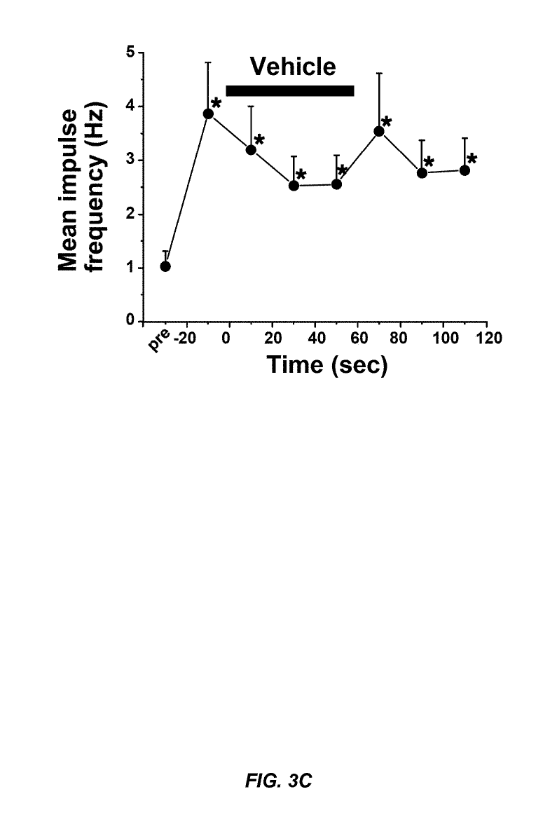

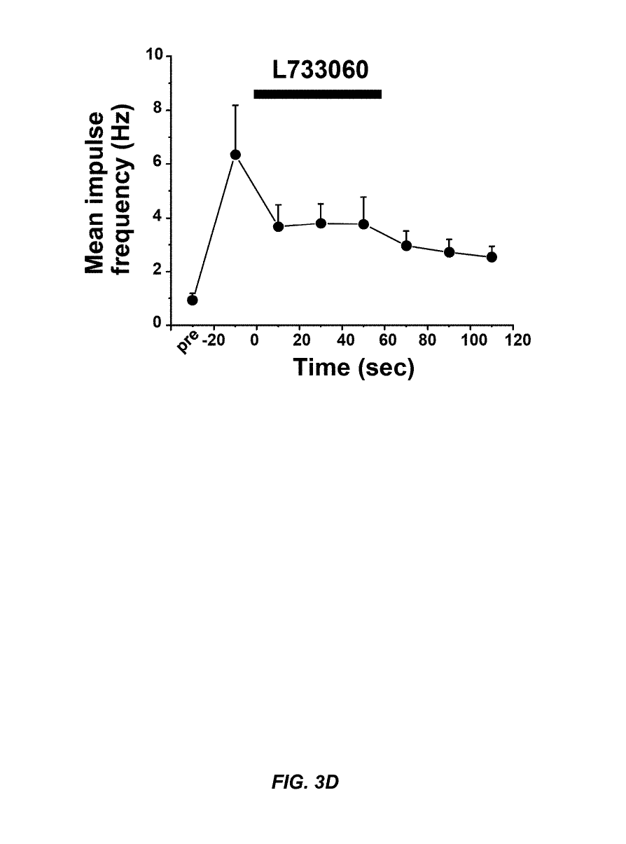

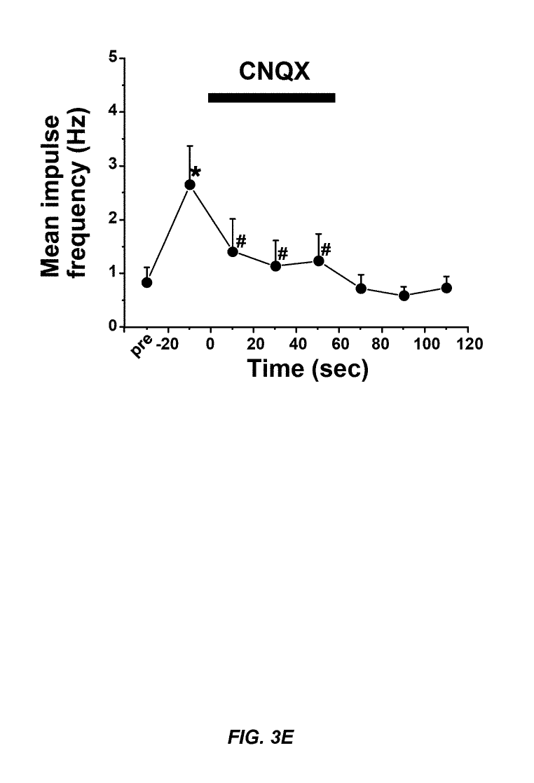

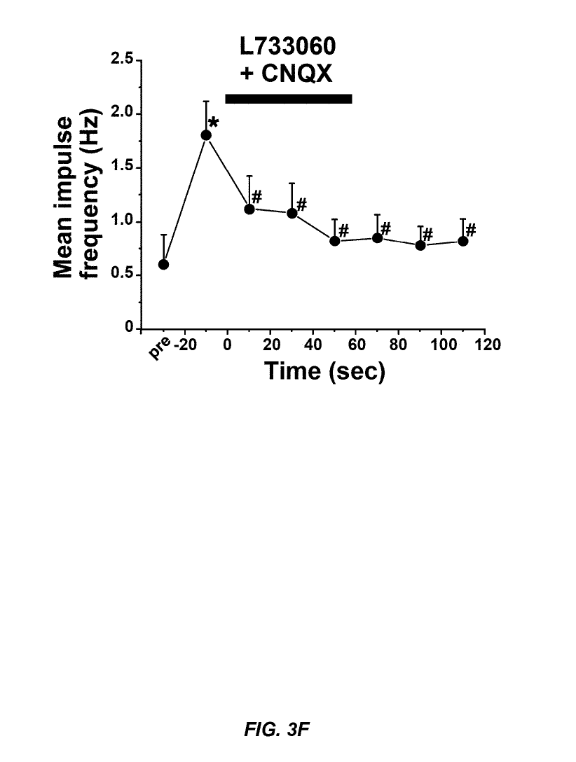

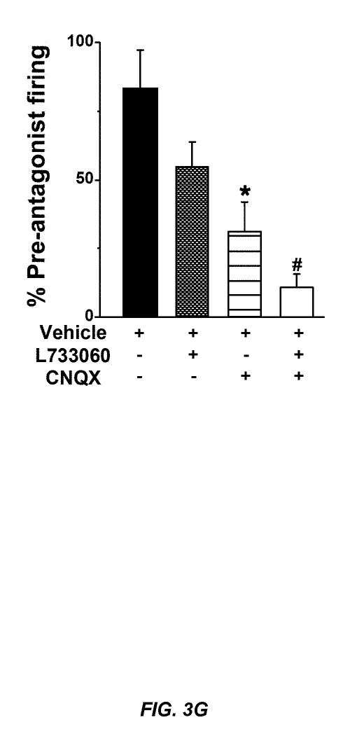

As a positive control, we examined the relationship between spinal superfusion with CNQX and reduced responses of dorsal horn neurons to noxious stimuli as previously reported [Dougherty P et al., The Journal of neuroscience: the official journal of the Society for Neuroscience 12(8):3025-3041 (1992); Fume H et al., The Journal of physiology 521 Pt 2:529-535 (1999); King A and Lopez-Garcia J., The Journal of physiology 472:443-457 (1993); Schneider S and Perl E., Journal of neurophysiology 72(2):612-621 (1994)]. Topical AITC elicited a significant increase in firing of superficial dorsal horn neurons that declined slightly over the ensuing 2 minutes (FIG. 3A, 3C). The NK-1 receptor antagonist numerically reduced AITC-evoked firing (FIG. 3D, 3G). CNQX significantly reduced AITC-evoked firing (FIG. 3E, 3G; p<0.05). Superfusion with both L733060 and CNQX further reduced AITC-evoked firing (FIG. 3B, F) to a level that was significantly different compared to either individual antagonist (FIGS. 3F, 3G; p<0.001 vs. vehicle).

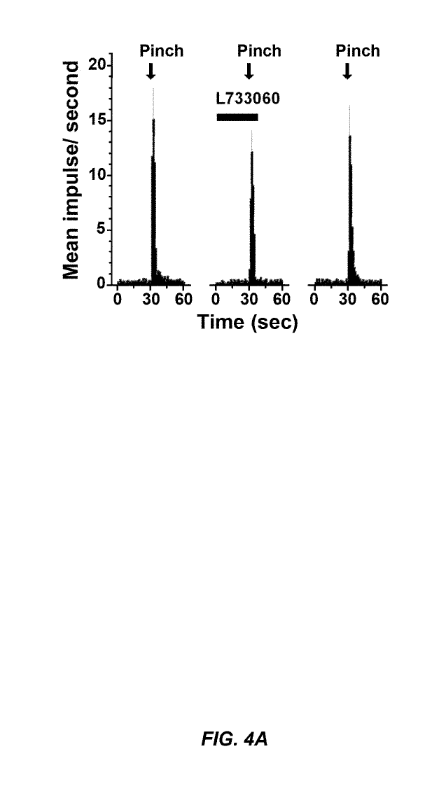

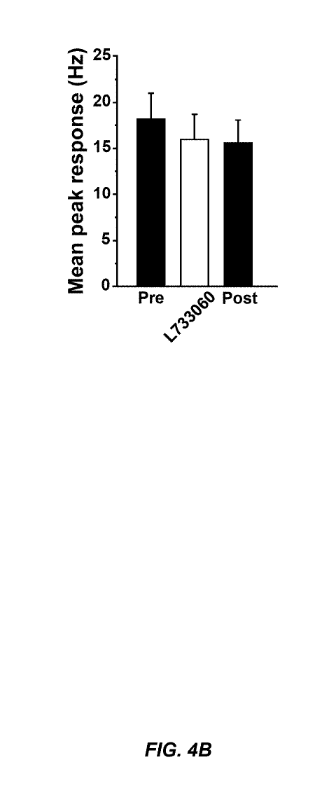

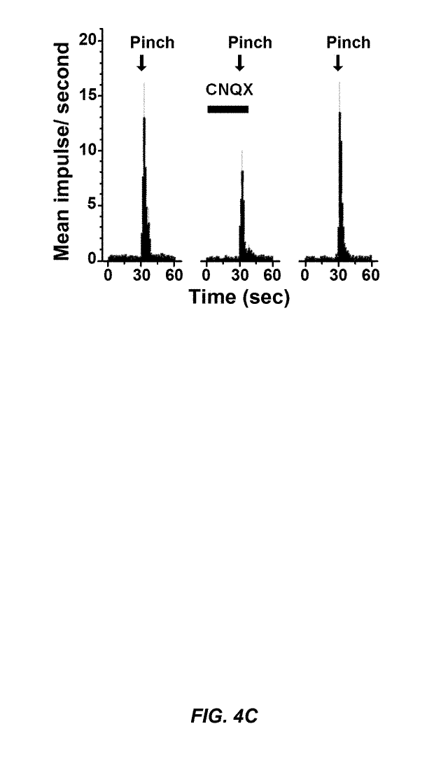

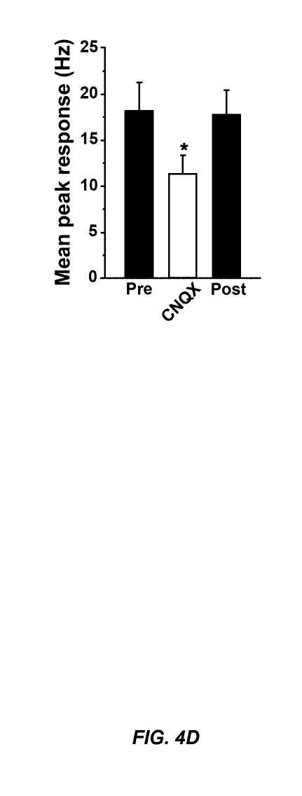

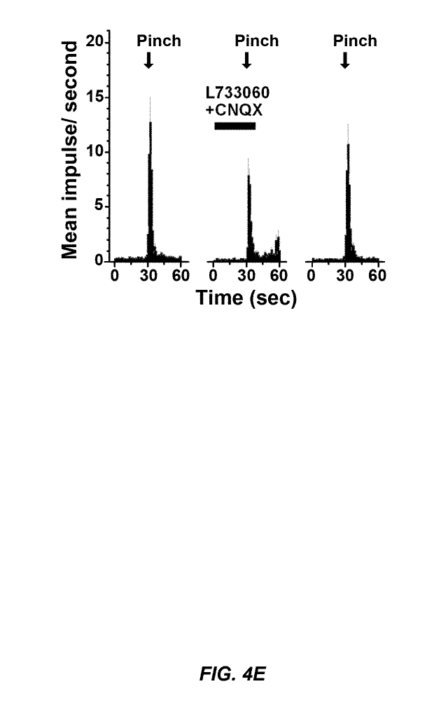

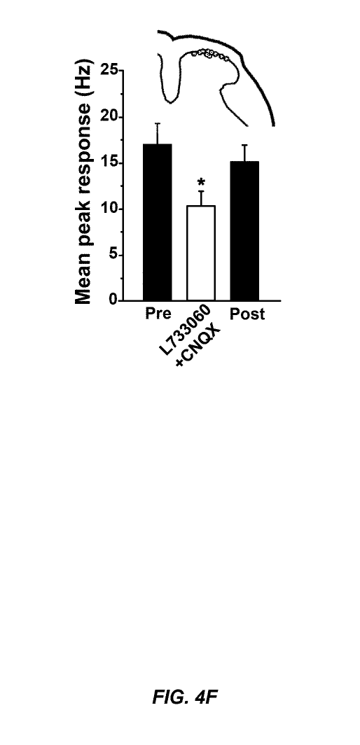

We additionally tested the effects these antagonists on spinal unit responses to noxious pinch. We tested chloroquine- and pinch-responsive units, as well as separate populations of NS and WDR units that were isolated by their response to pinch and that were unresponsive to id chloroquine. A total of 22 units was tested with the NK-1 antagonist (7 pinch+chloroquine responsive, 15 pinch but not chloroquine responsive), 39 with the AMPA/kainite antagonist (24 pinch+chloroquine-responsive; 15 pinch but not chloroquine responsive) and 45 with both antagonists (23 pinch+chloroquine responsive, 22 pinch but not chloroquine responsive). Effects of antagonists on pinch-evoked responses were very similar for chloroquine-responsive and -unresponsive units, and for NS and WDR units, so data were pooled. Noxious pinch elicited a transient robust increase in firing that was not reduced by the NK-1 receptor antagonist (FIG. 4A, B), but was significantly reduced by CNQX, followed 30 minutes later by recovery (FIG. 4C, D; p<0.001). Co-application of the NK-1 antagonist and CNQX did not reduce the pinch-evoked response to any greater extent compared to CNQX alone (FIG. 4E, 4F; p<0.001 vs. pre). Those data are consistent with previous studies [De Koninck Y and Henry J L, Proceedings of the National Academy of Sciences of the United States of America 88(24):11344-11348 (1991); Dougherty P et al., The Journal of neuroscience: the official journal of the Society for Neuroscience 12(8):3025-3041 (1992); Dougherty P et al., Journal of neurophysiology 72(4):1464-1475 (1994); Fume H et al., The Journal of physiology 521 Pt 2:529-535 (1999); King A and Lopez-Garcia J., The Journal of physiology 472:443-457 (1993); Rees H et al., Experimental brain research Experimentelle Hirnforschung Experimentation cerebrate 121(3):355-358 (1998); Schneider S and Perl E., Journal of neurophysiology 72(2):612-621 (1994)].

### Behavior

Chloroquine-Evoked Scratching was Inhibited More by Antagonist Co-Application.

Intrathecal administration of each antagonist significantly attenuated chloroquine-evoked scratching (FIG. 5; p<0.005). Co-administration of the NK-1 antagonist and CNQX attenuated chloroquine-evoked scratching to a greater extent than either antagonist individually (FIG. 5). Combined it co-application of all three antagonists (GRPR, NK-1 and AMPA) reduced scratching to an even greater extent compared to co-application of the NK-1 antagonist and CNQX (FIG. 5).

CNQX Reduced Histamine-Evoked Scratching Behavior.

Intrathecal administration of neither the NK-1 nor the GRPR antagonist attenuated histamine-evoked scratching (FIG. 6). In contrast, the number of histamine-evoked scratch bouts was significantly decreased by CNQX (FIG. 6; p<0.05) to a level that did not differ from that elicited by id injection of vehicle (10.1±4.0; p=0.202 unpaired t-test) [Akiyama T et al., The Journal of investigative dermatology 132(7):1886-1891 (2012)].

### Cell Imaging

Calcium Imaging of DRG Cells.

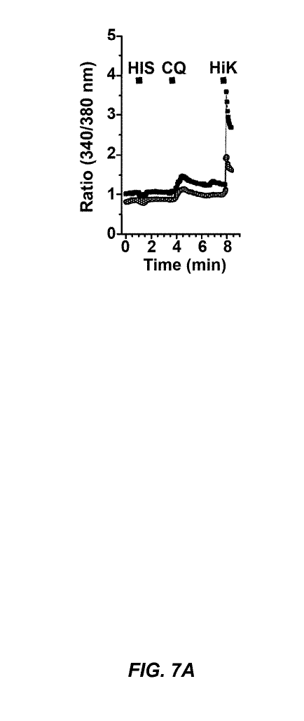

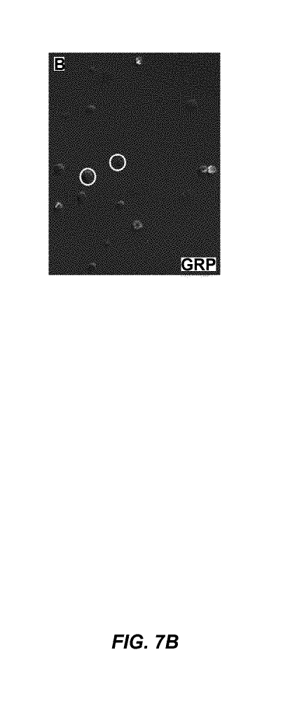

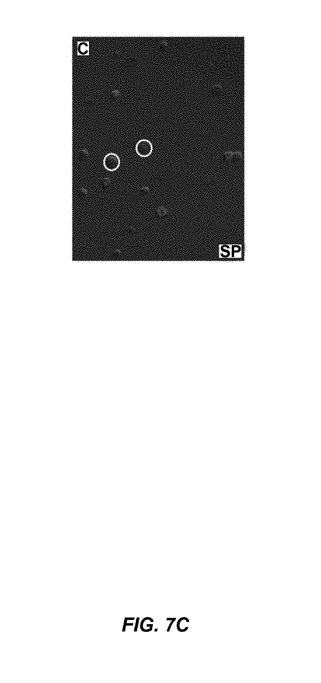

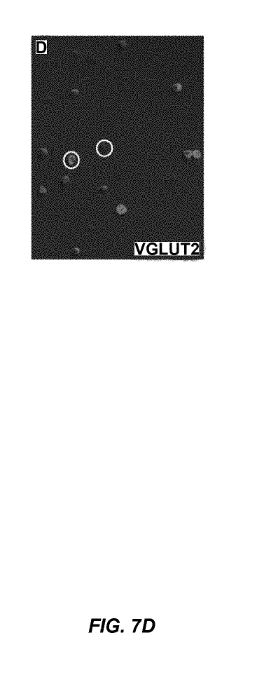

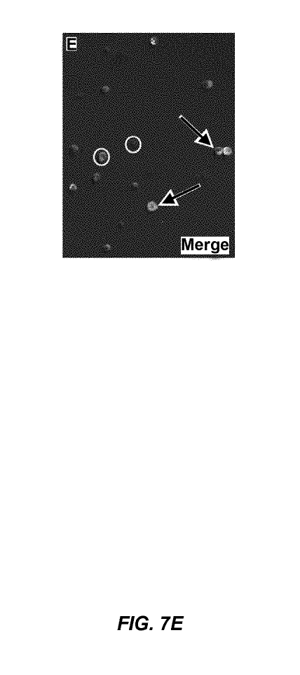

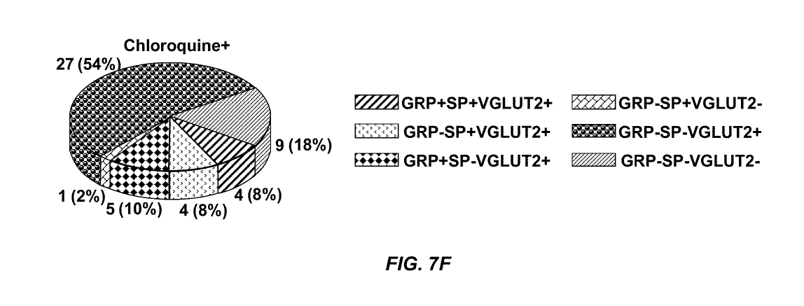

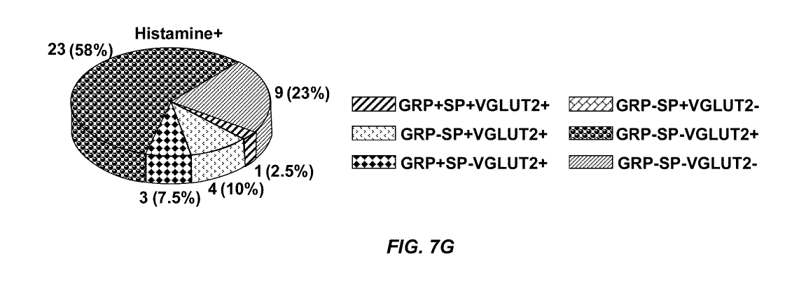

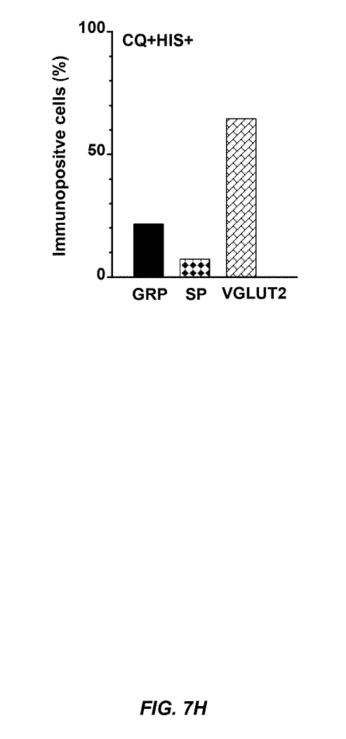

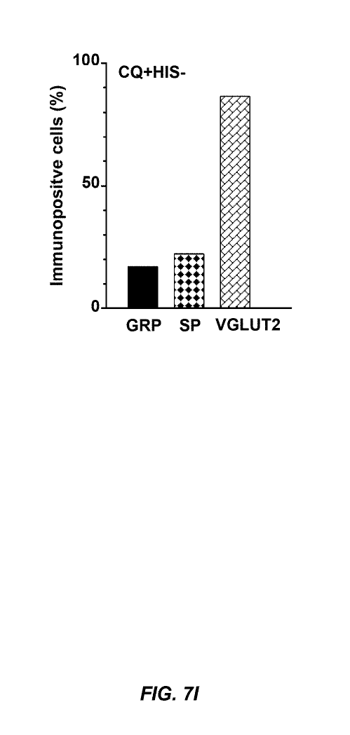

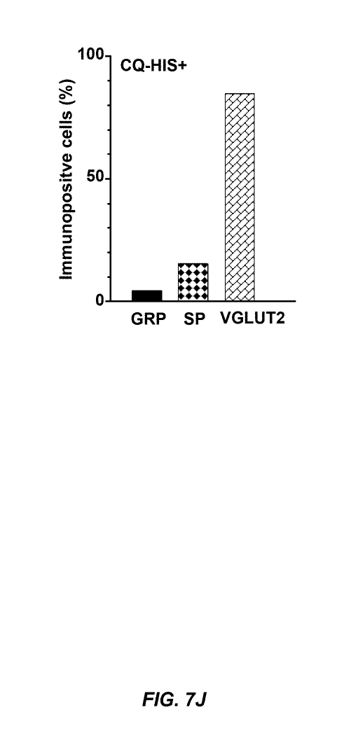

Of a total of 898 DRG cells imaged, 8.4% responded to chloroquine and 12.9% responded to histamine, consistent with previous studies [Akiyama T et al., Pain 151(2):378-383 (2010); Akiyama T et al., Journal of neurophysiology 109(3):742-748 (2013); Liu Q et al., Cell 139(7):1353-1365 (2009); Wilson S R et al., Nature neuroscience 14(5):595-602 (2011)]. FIG. 7A shows examples of two DRG cells that responded to chloroquine but not histamine.

Immunofluorescent Labeling of Pruritogen-Responsive DRG Cells.

Following calcium imaging, the DRG cells were fixed and triple-immunostained for SP, GRP and VGLUT2. A total of 597 DRG cells was labeled. FIG. 7B-7E show immunostained DRG cells from the calcium imaging experiment shown in FIG. 7A; the two chloroquine-responsive cells in A are indicated by circles. FIG. 7B shows GRP-immunopositive DRG cells. FIG. 7C shows DRG cells immunopositive for SP, including one of the chloroquine-responsive cells exhibiting weak immunoreactivity. FIG. 7D shows cells immunopositive for VGLUT2, including one of the chloroquine-responsive cells. The merged view in FIG. 7E shows cells that were triple-labeled (arrows) for VGLUT2, SP and GRP.

Overall, 27.1% of all DRG cells examined were immunopositive for SP, 23.8% for GRP, and 79.2% for VGLUT2, consistent with previous studies [Akiyama T et al., Journal of neurophysiology 109(3):742-748 (2013); Caterina M J et al., Science 288(5464):306-313 (2000); Chen C L et al., Neuron 49(3):365-377 (2006); Dirajlal S et al., Journal of neurophysiology 89(1):513-524 (2003); Scherrer G et al., Proceedings of the National Academy of Sciences of the United States of America 107(51):22296-22301 (2010); Sun Y G and Chen Z F, Nature 448(7154):700-703 (2007); Tominaga M et al., The Journal of investigative dermatology 129(12):2901-2905 (2009)]. Of the DRG cells that responded to chloroquine, 16%, 18% and 80% were immunpositive for SP, GRP and VGLUT2, respectively (FIG. 7F). Of the histamine-responsive cells, 10%, 17.5% and 77% were immunpositive for SP, GRP and VGLUT2, respectively (FIG. 7G). All of the histamine-responsive cells that were immunopositive for either SP or GRP were also immunopositive for VGLUT2 (FIG. 7G). FIG. 7H shows the incidence of immunostaining of DRG cells that responded to both chloroquine and histamine; more cells co-expressed GRP (21%) than SP (7%). FIG. 7I similarly shows the incidence of immunostaining of DRG cells that responded to chloroquine but not histamine, of which GRP and SP were approximately equally co-expressed (GRP 17%; SP 22%). FIG. 7J shows the incidence of immunostaining of DRG cells that responded to histamine but not chloroquine. Of these cells, SP was predominantly expressed (15%) compared to GRP (4%).

### Discussion

The present findings indicate that non-histaminergic itch is mediated by the combined intraspinal release of glutamate, SP and GRP from chloroquine-sensitive pruriceptors to activate itch-signaling spinal neurons. In contrast, histamine-mediated itch depends primarily on glutamate, with GRP playing a lesser role in NS neurons. These conclusions are supported by the following: (1) Chloroquine-evoked responses were suppressed by each individual antagonist and completely inhibited by co-application, while CNQX alone abolished histamine-evoked responses. (2) Behavioral studies provided comparable results. (3) Ten-18% of chloroquine- or histamine-sensitive DRG neurons co-expressed substance P or GRP while ˜80% co-expressed VGLUT2. These data additionally support the concept of co-administration of AMPA/kainate, NK-1 and GRPR antagonists to treat itch, including chronic itch, antihistamine-resistant, and antihistamine-sensitive itch. The NK-1 antagonist aprepitant shows promise for chronic itch [Stander S et al., PloS one 5(6):e10968 (2010)]. An AMPA antagonist (Perampanel) is FDA-approved for epilepsy, and a GRPR antagonist shows promise in treating cancer [Schwartsmann G et al., Investigational new drugs 24(5):403-412 (2006)].

### Role of Chloroquine-Responsive Dorsal Horn Neurons in Itch

Chloroquine acts at MrgprA3 expressed in primary afferent C-fibers [Liu Q et al., The Journal of neuroscience: the official journal of the Society for Neuroscience 32(42):14532-14537 (2012)]. MrgprA3-expressing afferents responded to chloroquine, and histamine, capsaicin and other noxious stimuli [Han L et al., Nature Neuroscience 16(2):174-82 (2013)], similar to the present and previously-recorded [Akiyama T et al., Program No 37511/PP82012 Neuroscience Meeting Planner New Orleans, La.: Society for Neuroscience, online 2012] dorsal horn neurons. In mice lacking the capsaicin-sensitive ion channel TRPV1, TRPV1 was selectively re-expressed in MrgprA3-expressing DRG neurons [Han L et al., Nature Neuroscience 16(2):174-82 (2013)]. In these animals, id injection of capsaicin, which normally elicits nocifensive wiping behavior, instead elicited hindlimb scratching indicative of itch [Han L et al., Nature Neuroscience 16(2):174-82 (2013)]. This implies that MrgprA3-expressing primary afferent fibers are linked to a “labeled line” itch pathway, regardless of what type of stimulus activates them. Accordingly, it is believed that the chloroquine-responsive dorsal horn neurons recorded presently contribute to neural circuits that selectively signal itch and generate scratching behavior.

### Spinal Neurotransmitters Mediating Histaminergic and Non-Histaminergic Itch

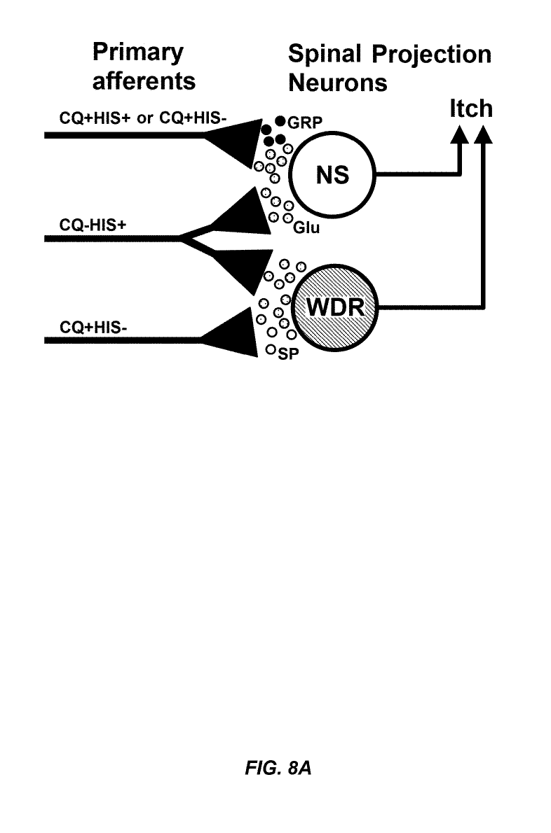

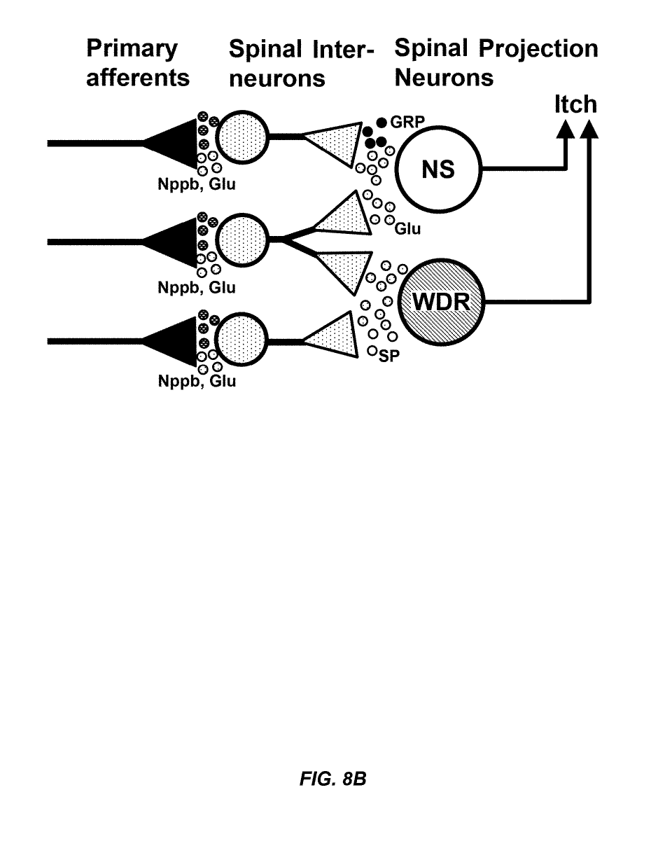

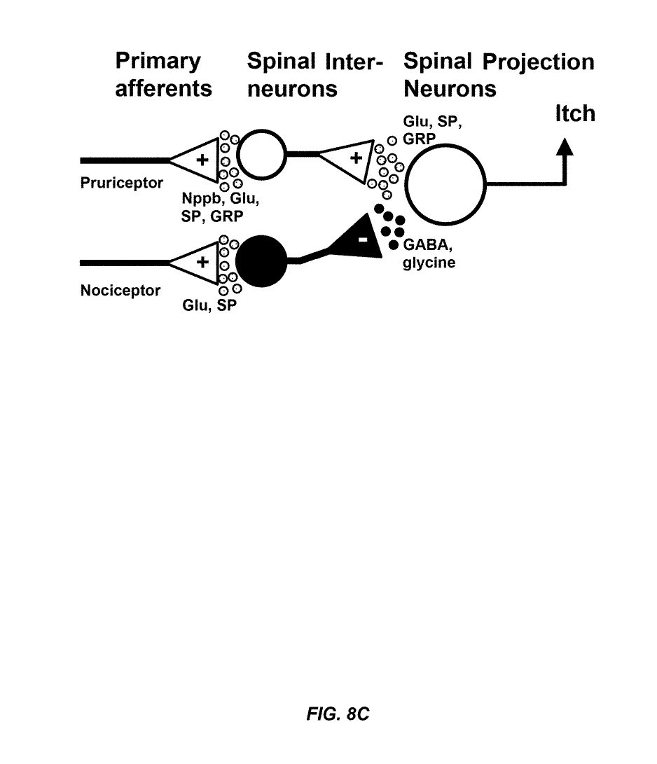

Chloroquine-evoked scratching and spinal neuronal firing was reduced by individually-applied GRPR, NK-1 (except for NS neurons) or AMPA antagonists, and was abolished by their co-application. It is believed that GRP, SP and glutamate are released from intraspinal terminals of chloroquine-sensitive pruriceptors to excite itch-signaling dorsal horn neurons (FIG. 8A). Consistent with this, chloroquine-sensitive DRG cells co-expressed GRP, SP and VGLUT2. In contrast, CNQX inhibited histamine-evoked neuronal responses, with the NK-1 and GRPR antagonists having lesser or no effect, implying that glutamate is the primary spinal neurotransmitter for histaminergic itch.

Most chloroquine-responsive neurons also responded to histamine. It is novel that responses of the same neuron to chloroquine and histamine were pharmacologically distinct. Chloroquine- and/or histamine-sensitive pruriceptors consist of functionally distinct subpopulations and are thought to release different neurotransmitters [Roberson D P et al., Nature neuroscience 16(7):910-918 (2013)]. Chloroquine excitation of NS neurons was reduced by NK-1 and AMPA antagonists (FIG. 1J), implying input primarily from pruriceptors that release SP and glutamate, i.e., the chloroquine-sensitive histamine-insensitive (CQ+HIS−) and chloroquine-insensitive histamine-sensitive (CQ−HIS+) DRG cells (FIG. 6). Chloroquine-evoked excitation of WDR neurons was reduced by GRPR and AMPA antagonists, implying input from pruriceptors that release GRP (i.e., CQ+HIS+ and CQ+HIS−; FIG. 8A). Histamine-evoked excitation of both WDR and NS cells was inhibited by CNQX, implying input primarily from histamine-sensitive pruriceptors (i.e., CQ−HIS+ and CQ+HIS+). That the GRPR antagonist partly inhibited responses of NS (but not WDR) neurons (FIG. 2) implies input from chloroquine- and histamine-sensitive (CQ+HIS+) DRG cells containing GRP (FIG. 8A). The lack of effect of the NK-1 antagonist on histamine-evoked responses implies that NS and WDR neurons either do not receive input from cells that co-express SP (CQ−HIS+), or that they do but SP participates exclusively in a peripheral role such as the axon reflex induced by histamine [Schmelz M et al., Neuroreport 11(3):645-648 (2000)].

Another explanation for the pharmacologically distinct effects of chloroquine and histamine is the firing pattern of primary afferent fibers. MrgprA3-expressing sensory neurons exhibited distinct firing patterns (bursting vs. steady state) to different itch mediators [Han L et al., Nature neuroscience 16(2):174-182 (2013)] that may determine whether glutamate or a neuropeptide is released from the presynaptic terminal.

A third possibility is that NS and WDR neurons receive inputs from different populations of interneurons using different neurotransmitters (FIG. 8B). Natriuretic polypeptide b (Nppb) is released from central terminals of primary afferents to excite GRPR-expressing spinal interneurons, a pathway that accounts for all pruritogen-evoked scratching behavior in mice [Mishra S K and Hoon M A, Science 340(6135):968-971 (2013)]. The present data are not inconsistent with this, since SP, GRP and glutamate are expressed in spinal interneurons [Todd A J et al., The European journal of neuroscience 17(1):13-27 (2003); Wang X et al., Neuron 78(2):312-324 (2013)].

We confirmed expression of SP in a subpopulation of chloroquine-sensitive DRG cells [Akiyama T et al., Journal of neurophysiology 109(3):742-748 (2013)]. However, most MrgprA3-expressing sensory neurons co-expressed calcitonin gene-related peptide (CGRP) and IB-4, but not SP [Han L et al., Nature neuroscience 16(2):174-182 (2013)]. Chloroquine-evoked scratching was reduced by ˜70% in mice lacking MrgprA3 [Han L et al., Nature neuroscience 16(2):174-182 (2013)]; residual scratching may be mediated by SP-expressing, MrgprA3-negative neurons. SP is presumably released by a small subpopulation of non-histaminergic pruriceptors [Song X and Zhao Z, Neuroscience letters 168(1-2):49-52 (1994)]. In contrast, NK-1 antagonists failed to inhibit histamine-evoked scratching or neuronal firing, even though SP was expressed in some histamine-sensitive chloroquine-insensitive DRG neurons. That all neuropeptide-expressing histamine-sensitive DRG neurons co-expressed VGLUT2 indicates that glutamate is the main neurotransmitter released by histamine-sensitive pruriceptors. Consistent with this, electrical stimulation of dorsal roots evoked responses in histamine-sensitive spinal neurons that were abolished by CNQX [Koga K et al., Molecular pain 7:47 (2011)].

We confirm that GRP was coexpressed by some chloroquine-sensitive neurons [Akiyama T et al., Journal of neurophysiology 109(3):742-748 (2013)]. Chloroquine-evoked scratching and spinal neuronal firing were reduced by the GRPR antagonist, which did not affect histamine-evoked scratching (but reduced responses of NS neurons). Activity in the unaffected WDR neurons may have compensated for inhibition of NS neurons to account for the lack of GRPR antagonist effect on scratching.

The specificity of the GRP antibody and presence of GRP within DRG neurons has been questioned [Liu Y et al., Neuron 68(3):543-556 (2010)], although GRP mRNA and protein were reported to be expressed in substantial populations of primary sensory neurons [Akiyama T et al., Journal of neurophysiology 109(3):742-748 (2013); Alemi F et al., The Journal of clinical investigation 123(4):1513-1530 (2013); Fleming M et al., Molecular pain 8:52 (2012); Lagerstrom M C et al., Neuron 68(3):529-542 (2010); Liu Q et al., Cell 139(7):1353-1365 (2009); Liu T et al., The Journal of clinical investigation 122(6):2195-2207 (2012); Liu T et al., Nature neuroscience 13(12):1460-1462 (2010); Liu Y et al., Neuron 68(3):543-556 (2010); Sun Y G and Chen Z F, Nature 448(7154):700-703 (2007); Tominaga M et al., The Journal of investigative dermatology 129(12):2901-2905 (2009)]. However, GRP staining of the rhizotomized spinal cord revealed that most GRP is synthesized locally [Fleming M et al., Molecular pain 8:52 (2012)]. Thus, GRP may be released primarily by spinal interneurons (FIG. 8B), rather than (or in addition to) its release from pruriceptors. In this scenario, spinal neurons sensitive to GRP may be synaptically excited by glutamate, rather than GRP, released from primary afferents [Koga K et al., Molecular pain 7:47 (2011)].

Most pruritogen-responsive DRG neurons expressed VGLUT2, implying that pruriceptors release glutamate. Knockout mice lacking VGLUT2 in primary afferents exhibited reduced nociception and enhanced scratching, explained by decreased release of glutamate from nociceptors and reduced excitation of inhibitory spinal interneurons, thereby disinhibiting itch-signaling neurons [Lagerstrom M C et al., Neuron 68(3):529-542 (2010); Liu Y et al., Neuron 68(3):543-556 (2010); Ross S E et al., Neuron 65(6):886-898 (2010)]. FIG. 8C shows a balance of excitatory pruriceptive and inhibitory interneuronal inputs onto itch-signaling spinal neurons. It is believed that knockout of VGLUT2 more strongly reduces nociceptive than pruriceptive afferent drive, shifting the balance toward enhanced itch transmission. Antagonism of spinal AMPA receptors more strongly blocks direct and indirect (via excitatory interneurons) glutamatergic pruriceptive input, shifting the balance toward reduced itch transmission.

### Spinal Nociceptive Transmission

Noxious thermal, mechanical and chemical stimuli evoked spinal release of SP [Duggan A W et al., Brain Res 451(1-2):261-273 (1988)] which elicited prolonged excitatory postsynaptic potentials (EPSPs) [Chapman V and Dickenson A, Neuroscience letters 157(2):149-152 (1993); Urbán L and Randić M, Brain research 290(2):336-341 (1984)] that were inhibited by an NK-1 antagonist [De Koninck Y and Henry J L, Proceedings of the National Academy of Sciences of the United States of America 88(24):11344-11348 (1991)]. Spinal neurons exhibiting prolonged mechanically-evoked EPSPs had greater numbers of synaptic contacts by SP-immunoreactive varicosities [De Koninck Y et al., Proceedings of the National Academy of Sciences of the United States of America 89(11):5073-5077 (1992)]. Spinal neuronal responses to formalin and capsaicin, were inhibited by NK-1 antagonists [Chapman V and Dickenson A, Neuroscience letters 157(2):149-152 (1993); Dougherty P et al., Journal of neurophysiology 72(4):1464-1475 (1994); Rees H et al., Experimental brain research Experimentelle Hirnforschung Experimentation cerebrale 121(3):355-358 (1998)]. Consistent with this, the NK-1 antagonist presently inhibited dorsal horn neuronal responses to AITC but not noxious pinch. The lack of effect of the NK-1 antagonist on pinch-evoked responses is consistent with previous studies [De Koninck Y and Henry J L, Proceedings of the National Academy of Sciences of the United States of America 88(24):11344-11348 (1991)] [Dougherty P et al., Journal of neurophysiology 72(4):1464-1475 (1994); Mazario J and Basbaum A I, The Journal of neuroscience: the official journal of the Society for Neuroscience 27(4):762-770 (2007); Rees H et al., Experimental brain research Experimentelle Hirnforschung Experimentation cerebrale 121(3):355-358 (1998)]. Mice with genetic ablation of non-peptidergic MrgprD-expressing sensory neurons exhibited reduced behavioral responses to noxious mechanical but not thermal stimuli, suggesting a role for non-peptidergic sensory neurons in mechanical nociception [Cavanaugh D et al., Proceedings of the National Academy of Sciences of the United States of America 106(22):9075-9080 (2009)].

Glutamate is generally associated with spinal nociceptive transmission. Spinal neuronal responses to pinch and AITC were presently reduced or blocked by CNQX, consistent with previous studies [Dougherty P et al., The Journal of neuroscience: the official journal of the Society for Neuroscience 12(8):3025-3041 (1992); Fume H et al., The Journal of physiology 521 Pt 2:529-535 (1999); King A and Lopez-Garcia J., The Journal of physiology 472:443-457 (1993); Schneider S and Perl E., Journal of neurophysiology 72(2):612-621 (1994)]. The present results indicate that spinal pathways signaling itch and pain share glutamate and SP as excitatory neurotransmitters. This is consistent with previous studies showing that most pruritogen-sensitive spinal neurons also respond to algogens, suggesting that the central nervous system discriminates between itch and pain based on input from partially overlapping subpopulations of itch- and pain-signaling neurons [Akiyama T et al., Journal of neurophysiology 102(4):2176-2183 (2009); Akiyama T et al., Journal of neurophysiology 104(5):2442-2450 (2010); Akiyama T et al., The Journal of neuroscience: the official journal of the Society for Neuroscience 29(20):6691-6699 (2009); Davidson S et al., Journal of neurophysiology 108(6):1711-1723 (2012); [17] Davidson S et al., The Journal of neuroscience: the official journal of the Society for Neuroscience 27(37):10007-10014 (2007)].

1-16. (canceled)

17. A method of inhibiting spinal neurotransmission of itch comprising administering to a subject suffering from itch:
i) a first inhibitor of spinal neurotransmission of itch signal comprising:
a) an NK-1 receptor antagonist selected from the group consisting of LY733060, aprepitant, AV608, GR205171, GW679769, LY686017, L-759274, orvepitant, vestipitant, GSK206136, GW67969, GSK1144814, SSR20600C, and nolpitatium besylate; and/or
(b) a GRP receptor antagonist; and
ii) a second inhibitor of spinal neurotransmission of itch signal comprising an AMPA glutamate receptor antagonist selected from the group consisting of CNQX, talampanel, ZK 200775, GSK729327, topiramate, LY300164, and BGG492.

18. The method of claim 17, wherein the GRP receptor antagonist is RC-3095.

19. The method of claim 17, wherein the AMPA glutamate receptor antagonist is CNQX.

20. The method of claim 17, wherein the method comprises administering to the subject:
the NK-1 receptor antagonist, wherein the NK-1 receptor antagonist is LY733060;
the GRP receptor antagonist, wherein the GRP receptor antagonist is RC-3095; and
an AMPA glutamate receptor antagonist, wherein the AMPA glutamate receptor antagonist is CNQX.

21. The method of claim 17, wherein the step of administering comprises systemic, epidural, or intrathecal administration.

22. The method of claim 21, wherein systemic administration comprises intraperitoneal, subcutaneous, intravenous, oral, intradermal, or dermal administration.

23. A method of inhibiting spinal neurotransmission of itch comprising administering to a subject suffering from itch an AMPA glutamate receptor antagonist selected from the group consisting of CNQX, talampanel, ZK 200775, GSK729327, topiramate, LY300164, and BGG492; and:
an NK-1 receptor antagonist selected from the group consisting of LY733060, aprepitant, AV608, GR205171, GW679769, LY686017, L-759274, orvepitant, vestipitant, GSK206136, GW67969, GSK1144814, SSR20600C, and nolpitatium besylate, and/or
a GRP receptor antagonist, wherein the GRP antagonist is RC-3095.

24. A formulation comprising:
i) a first inhibitor of spinal neurotransmission of itch signal comprising:
a) an NK-1 receptor antagonist selected from the group consisting of LY733060, aprepitant, AV608, GR205171, GW679769, LY686017, L-759274, orvepitant, vestipitant, GSK206136, GW67969, GSK1144814, SSR20600C, and nolpitatium besylate; and/or
b) a GRP receptor antagonist, wherein the GRP receptor antagonist is RC-3095;
ii) a second inhibitor of spinal neurotransmission of itch signal comprising an AMPA glutamate receptor antagonist selected from the group consisting of CNQX, talampanel, ZK 200775, GSK729327, topiramate, LY300164, and BGG492; and
iii) a pharmaceutically acceptable excipient.

25. The formulation of claim 24, wherein i) comprises the NK-1 receptor antagonist selected from the group consisting of LY733060, aprepitant, AV608, GR205171, GW679769, LY686017, L-759274, orvepitant, vestipitant, GSK206136, GW67969, GSK1144814, SSR20600C, and nolpitatium besylate.

26. The formulation of claim 24, wherein i) comprises the GRP receptor antagonist RC-3095.

27. The formulation of claim 24, wherein the formulation comprises:
the NK-1 receptor antagonist selected from the group consisting of LY733060, aprepitant, AV608, GR205171, GW679769, LY686017, L-759274, orvepitant, vestipitant, GSK206136, GW67969, GSK1144814, SSR20600C, and nolpitatium besylate; and
the GRP receptor antagonist RC-3095.

28. The formulation of claim 24, wherein the formulation is a formulation for intrathecal administration.

29. The formulation of claim 24, wherein the NK-1 receptor antagonist comprises LY733060.

30. The formulation of claim 24, wherein the AMPA glutamate receptor antagonist comprises CNQX.

31. The formulation of claim 24, wherein the formulation comprises CNQX, and RC-3095 or LY733060.

32. The formulation of claim 24, wherein the formulation comprises RC-3095, LY733060, and CNQX.

33. The formulation of claim 27, wherein the formulation comprises aprepitant and RC-3095.

34. The method of claim 17, wherein the method comprises intrathecal administration of the GRP receptor antagonist and systemic, epidural, or intrathecal administration of the NK-1 receptor antagonist.

35. The method of claim 23, wherein the method comprises administering:
the AMPA glutamate receptor antagonist selected from the group consisting of CNQX, talampanel, ZK 200775, GSK729327, topiramate, LY300164, and BGG492; and
the NK-1 receptor antagonist selected from the group consisting of LY733060, aprepitant, AV608, GR205171, GW679769, LY686017, L-759274, orvepitant, vestipitant, GSK206136, GW67969, GSK1144814, SSR20600C, and nolpitatium besylate.

36. The method of claim 23, wherein the method comprises administering CNQX, and LY733060.
